SEQUENCE LISTING (13 sequences)

SEQ ID NO: 1 - protein, 5 aa, Artificial Sequence
  Synthetic Sequence
VGGGS

SEQ ID NO: 2 - protein, 5 aa, Artificial Sequence
  Synthetic Sequence
PSSNA

SEQ ID NO: 3 - protein, 5 aa, Artificial Sequence
  Synthetic Sequence
HPSLK

SEQ ID NO: 4 - protein, 5 aa, Artificial Sequence
  Synthetic Sequence
HPSLG

SEQ ID NO: 5 - protein, 5 aa, Artificial Sequence
  Synthetic Sequence
HPSLL

SEQ ID NO: 6 - protein, 5 aa, Artificial Sequence
  Synthetic Sequence
HPSLA

SEQ ID NO: 7 - protein, 8 aa, Artificial Sequence
  Synthetic Sequence
NPSHPLSG

SEQ ID NO: 8 - protein, 8 aa, Artificial Sequence
  Synthetic Sequence
NPSHPSLG

SEQ ID NO: 9 - protein, 4 aa, Artificial Sequence
  Synthetic Sequence
GGGS

SEQ ID NO: 10 - protein, 8 aa, Artificial Sequence
  Synthetic Sequence
GGGSGGGS

SEQ ID NO: 11 - protein, 4 aa, Artificial Sequence
  Synthetic Sequence
SSSS

SEQ ID NO: 12 - protein, 8 aa, Artificial Sequence
  Synthetic Sequence
SSSSSSSS

SEQ ID NO: 13 - protein, 5 aa, Artificial Sequence
  Synthetic Sequence
VSNQH

(19) United States
(12) Patent Application Publication
(10) Pub. No.: US 2015/0299255 A1
(43) Pub. Date: Oct. 22, 2015

(54) COMPOSITIONS AND METHODS FOR MODULATING INNATE AND ADAPTIVE IMMUNE SYSTEMS

(71) Applicant: Susavion Biosciences, Inc., Tempe, AZ (US)

(72) Inventors: Laura L. Eggink, Scottsdale, AZ (US); J. Kenneth Hoober, Phoenix, AZ (US)

(21) Appl. No.: 14/697,240

(22) Filed: Apr. 27, 2015

Related U.S. Application Data
(63) Continuation-in-part of application No. 14/101,334, filed on Dec. 9, 2013.
Application No. 14/101,334 is a continuation-in-part of application No. 13/287,102, filed on Nov. 1, 2011, now abandoned.
(60) Provisional application No. 61/409,044, filed on Nov. 1, 2010.

(51) Int. Cl. C07K 7/06; A61K 39/00

(52) U.S. Cl. CPC C07K 7/06; A61K 39/00

(57) ABSTRACT

Compositions and methods for modulating the innate and adaptive immune systems in a subject. The compositions and methods may inhibit the function of inhibitory receptors and enhance activity of activating receptors. The method typically includes the steps of: administering to the subject a composition having a therapeutic peptide or multivalent polypeptide having multiple copies of the therapeutic peptide in an amount sufficient to increase activity of the immune system in the subject. Compositions may include a carrier; at least one agent selected from the group consisting of: an anti-inflammatory agent, a cytotoxic T cell proliferation agent, and/or a NK cell proliferation agent; and a selected therapeutic peptide or multivalent polypeptide. The compositions may also include an immunoglobulin admixed therewith to enhance passive immunoprotection.

### CROSS REFERENCE TO RELATED APPLICATIONS

This application is a continuation-in-part of U.S. patent application Ser. No. 14/101,334, filed on Dec. 9, 2013, which is a continuation-in-Part of U.S. patent application Ser. No. 13/287,102, filed on Nov. 1, 2011, which claims the benefit of U.S. Provisional Application No. 61/409,044, filed Nov. 1, 2010, the contents of each of which are incorporated herein by reference in their entireties.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 2,516 byte ASCII (text) file named “Seq_List” created on Apr. 24, 2015.

### FIELD OF THE INVENTION

The present invention is directed to therapeutic peptides and their uses in modulating the innate and adaptive immune systems in a subject.

### BACKGROUND

### Current Therapeutic Approaches to Viral Infections

Viruses such as HIV-1 cause immunosuppressive diseases. The primary therapy against HIV infections is daily administration of a combination of anti-retroviral drugs that inhibit viral replication after entry into the cell and subsequent maturation. The most commonly used are nucleoside reverse transcriptase inhibitors, non-nucleoside reverse transcriptase inhibitors, integrase inhibitors, and protease inhibitors that block enzymatic processing of viral products. These drugs effectively inhibit replication of the virus inside an infected cell and reduce viral load in the blood to undetectable levels [1, 2].

Another therapeutic approach uses fusion inhibitors, including proteins (e.g. monoclonal antibodies), peptides and small molecule agents (e.g. drugs), some of which act on the outside of the cell to prevent HIV from binding to receptors and fusing with the cell membrane [3]. Fusion inhibitors effectively block infection by HIV-1 and significantly reduce the systemic viral load. However, sufficient quantities of these drugs must be present in patients continuously to compete with the virus for the receptors. Vaccines that elicit antibodies that inhibit such fusion are of interest in this regard, and great effort is being directed towards achieving this goal [4].

A particularly confounding aspect of HIV-1 infections is the establishment of latent reservoirs, in which the integrated provirus stage can remain dormant for long periods of time. Consequently, the virus cannot be completely cleared from an infected individual by current treatments. Upon discontinuation of anti-retroviral treatment, these reservoirs are activated and the virus “rebounds” to pretreatment levels within a few weeks [5]. The question of whether the provirus is indeed dormant or simply replicates at a very low level has not been completely resolved.

Recent advances in the treatment of the RNA hepatitis C virus (HCV) involve development of protease inhibitors that act in a similar manner as those used to treat HIV-1 infections [6]. For HCV, the protease inhibitors are added to the currently accepted drug regime of pegylated interferon-alpha and ribavirin. An antiviral drug, ganciclovir, a precursor of a nucleotide analog that inhibits the viral DNA polymerase, is the commonly used treatment for acute infections of CMV. In general, the course of these diseases is moderated by the strength of the immune system.

### An Alternative Approach to Therapy

In contrast to therapeutic approaches aimed at prevention or control of the disease by directly inhibiting a step in the viral replication cycle, as described above, reactivation of patients' immune system is an alternative therapy that holds promise for restoring health and productivity to an infected patient in a practical, cost-effective manner. This approach provides a general defense against diseases rather than a pathogen-specific treatment. As a result, an intense interest in immunotherapy, as indicated by the development of cytokine treatments, is leading to products that can stimulate or inhibit the immune system. One developmental cytokine/immunomodulator project for the treatment of HIV/AIDS, for example, identified two key peptides derived from Thymus Nuclear Protein (TNP) technology (Viral Genetics, Inc., Azusa, Calif.), These peptides occur naturally in a variety of mammals, including humans.

The role of cytokines in the inhibition of HIV infectivity, particularly interleukin-16 (IL-16), interleukin-8 (IL-8) and RANTES (Regulated upon Activation, Normal T-cell Expressed, and Secreted; also known as CCL5), is very important. Cytokines such as IL-16, IL-8 and RANTES, which have overlapping and complementary functions, can act to attenuate viral infection by competing with viral binding with the receptors and by interfering with viral entry into cells by down-regulating the receptors required for entry. Other cytokines such as interferons (e.g., IFN-α. and IFN-γ) act to reduce viral load by activating intracellular anti-viral enzymes and also by stimulating antibody-mediated phagocytosis.

Interleukins (IL's) and interferons (IFN's) are potent cellular stimulants that are released from a variety of cells in response to insult or injury. Consequently, these proteins have attracted intense interest as therapeutic agents. IL-16 is a natural ligand of CD4 and should compete with virus for binding to T cells. IL-21 is required to avoid depletion of CD8⁺ T cells and also essential to maintain immunity and resolve persistent viral infections [7-9]. Similar to general stimulants such as lipopolysaccharide (LPS), however, IL's and IFN's induce release of inflammatory cytokines. Therefore, when given at higher than normal endogenous concentrations, they often have substantial adverse effects, which can be life-threatening and may require inpatient treatment facilities. Similarly, levels of TNF-α, IL-β and IL-6 are directly correlated with the probability of death in humans. Moreover, production of recombinant IL's and IFN's and their application are very costly. Even lower-dosage immunostimulant treatments developed for out-patient use have lower success rates and are not suitable in some situations such as, for example, to extend remission from cancer therapy or control a disease such as HIV at a chronic level. In view of this, it appears that exogenous therapeutic agents such as large, intact cytokine molecules are not well suited for general therapeutic use.

Usually, infections are cleared by the immune system through (i) internalization of the pathogen and presentation of antigens to T and B cells by dendritic cells (DCs), (ii) generation of antibodies by B cells, (iii) lysis of pathogen-infected cells by NK cells and CD8⁺ cytotoxic T lymphocytes (CTL), and/or (iv) destruction of the virus by antibody-mediated phagocytosis. While neutralizing antibody responses are subject to pathogen escape, many non-neutralizing antibodies that nevertheless bind the pathogen are present in infected patients. Restoration of immune effector cell functions, in particular phagocytic activity, which can recognize the resulting antigen-antibody complexes and destroy the complexes by antibody (Fc)-mediated phagocytosis, may be applicable to the clearance of infections in general.

The cell types that have significant involvement in viral infections in addition to phagocytic cells are in particular, two subsets of the T cell population (CD3⁺ and CD8⁺), NK cells (CD56⁺) and CTLs (CD8⁺). These cells are able to kill virus-infected cells and cancer cells by antibody-dependent cellular cytotoxicity (ADCC) in addition to an ability to directly lyse infected cells. NK cells are an integral component of the innate immune system and are primarily responsible for killing virus-infected and cancer cells. NK cells and CTL kill their targets mainly by releasing cytotoxic molecules such as perforin, granzymes and granlysin, which are contained in intracellular granules. These molecules are released when these cells make contact with target cells that contain antigens on the surface of viral infected or cancer cells to which antibodies bind. Activated NK cells also release cytokines and chemokines such as IFN-γ that activates macrophages and drives differentiation of CD4⁺ T cells into type 1 (Th1) cells [10, 11].

Information relevant to attempts to address one or more of these problems can be found in the following references: U.S. Patent Publication No. 2007/0003542; U.S. Patent Publication No. 2006/0269519; U.S. Patent Publication No. 2004/0248192; P. W. Latham, 1999; Fatkenheuer et al., 2005; Stover et al., 2006; Cohen, 2007; GlaxoSmithKline, 2005a and GlaxoSmithKline, 2005b. Each one of these treatments referred to in these references, however, suffers from one or more of the following disadvantages:

1. the size or composition of the agent provides significant challenges to cost-effective synthesis and purification;

2. the agent is specific for particular pathogen and/or cell type, rendering them unsuitable for general therapeutic use;

3. treatment with the agent induces clinically deleterious side effects that can be life-threatening, such as inflammation or hepatotoxicity, and require inpatient treatment facilities;

4. termination of treatment is followed soon thereafter by an increased systemic viral load;

5. long term exposure to agent often leads to treatment-resistant pathogens;

6. lower-dosage treatments developed for out-patient use have lower success rates and are not suitable in some situations;

7. treatment is ineffective, impractical, or cost-prohibitive for a large proportion of patients;

8. development of therapeutic antibodies require considerable medical infrastructure;

9. treatment such as vaccines may be appropriate to prevent infection but not to treat those already infected and who have a suppressed immune system;

10. no beneficial synergy between the immunogenic response induced and the effects of other endogenous immunoregulators;

11. agent inhibits the release of inhibitory cytokines that suppress release of beneficial cytokines, an indirect treatment; and

12. agent acts to restore baseline cytokine levels to balance responses of the immune system rather than promoting activation of phagocytes.

Many of these therapeutic protocols also become ineffective with time because mutation of the pathogen allows it to escape the treatment. Moreover, any immunosuppression that accompanies the disease attenuates the ability of the innate and adaptive immune systems to respond to antigenic changes and thereby keep the infection under control.

The immune system in individuals infected with a pathogenic agent such as HIV-1 initiates a defense response by production of antibodies. Even though the virus may mutate at one or a few sites and thereby escape the neutralizing activity of antibodies, endogenously produced non-neutralizing antibodies are usually polyclonal and may still bind the virus. The presence of anti-HIV-1 antibodies is often used as a diagnostic test for infection. During the course of the disease, the antibody level remains high whereas the ability to maintain a minimal viral load gradually weakens as the population of CD4⁺ T cells declines. The cellular components of the innate and adaptive immune response then become absent or quiescent. When the immune defense mechanisms reach a sufficiently low level, viral replication is not held in check and rapidly leads to a final stage of the disease, designated AIDS. However, even at this late stage, patients can be rescued from death by aggressive anti-retroviral therapy. Therefore, an agent that reactivates cells of the immune system, in particular phagocytes and NK cells, will likely also restore an immune defense against progression of the disease.

Not only is it essential to overcome the suppressive mechanisms of the pathogen, it is also important to modulate the host's natural mechanism to suppress the immune system. Therapeutic agents that activate/reactivate the immune system show particular promise in this regard, including cytokines and immunomodulators, although therapies based on exogenous agents such as large, intact cytokine molecules are not generally well suited for therapeutic use. Peptides, however, are often much more suitable therapeutic agents than large polypeptides or proteins. Peptides can, for example, be designed to induce one or more particular desired effects in vitro or in vivo, often without concomitantly inducing deleterious effects, and can usually be synthesized in a cost effective manner.

The development of this technology is applicable to diseases caused by other viruses, bacteria, fungi and cancers.

### Overcoming Immune System Suppression

Inhibitory mechanisms naturally maintain a balance within the immune system to prevent progression of an over-stimulated, deleterious immune response. Major components in these inhibitory mechanisms are cell-surface, lectin-type receptors of the siglec (sialic acid-binding immunoglobulin-like lectins) family [12]. The cytoplasmic domains of most siglecs contain ITIMs (immunoreceptor tyrosine-based inhibitory motifs) that when phosphorylated recruit tyrosine phosphatases such as SHP-1. Siglecs are abundant cell-surface proteins and bind to sialic acid residues on glycoprotein subunits of activating receptor complexes such as the B cell receptor (BCR). Siglec-associated SHP-1 then dephosphorylates (deactivates) the activating complexes, which suppresses immune functions [12, 13].

Most cells of the immune system contain several siglecs [12, 13]. A total of 14 siglecs is expressed in humans. As an example, the extensively studied Siglec-2 binds to sialic acid residues on BCR-associated proteins such as IgM, causes suppression of the activity of BCR in antigen recognition and antibody production [13, 14]. Siglec-2 contains six tyrosine residues within its cytoplasmic domain, three of which are within three ITIMs, that are potentially phosphorylated. Sialosides with high affinity to Siglec-2 bind to the sialic acid-binding site of Siglec-2 and release it from the BCR complex. Consequently, activation of the BCR complex is no longer attenuated and B cell activation ensues. It can be expected that similar suppression of other immune cells by siglecs is also relieved by binding of small molecules that inhibit the suppressive activity of these receptors. Whereas most siglecs express inhibitory functions, Siglec-14, -15 and -16 lack cytoplasmic ITIMs and serve activating functions in association with an adaptor protein DAP12, which contains an ITAM (immunoreceptor tyrosine-based activating motif) [12]. A compound that binds to multiple siglecs should provide a powerful mechanism to achieve immune cell activation by decreasing the suppressive ability of siglecs while also increasing activating functions. An additional receptor that binds sialic acid-galactose sequences is the potent activating receptor, NKG2D, which is expressed on NK cells, γδ T cells and CD8⁺ cytotoxic T cells [15]. NKG2D also functions with cytoplasmic adaptor proteins DAP10 and DAP12 that contain ITAMs. Peptides described in this invention are mimetics of sialic acid-galactose sequences and bind with high avidity to these receptors.

The peptides of this invention such as NPSHPLSG (SEQ ID NO:7) have a dual function. Firstly, they bind to the inhibitory siglec receptors. Secondly, they also bind to the activating receptor, NKG2D. The siglecs, as inhibitory receptors that prevent excessive activation of the immune system, are ‘checkpoints’. By binding to and inactivating these receptors, the peptides of this invention can be considered ‘checkpoint inhibitors’. Concomitantly, by binding to activating receptors on the same cell, the peptides exert a strong stimulatory effect on these cells. Essential for this function is the much greater avidity of the peptides to the sialic acid-binding sites than natural, cell-based glycans. In fact, siglecs bind sialic acid-containing ligands with K_{D} values in the low millimolar range, whereas the peptides bind with three orders of magnitude greater avidity, with K_{D} values of 1 micromolar or less. Thus the peptides are well suited to provide therapeutic benefit.

Current immunotherapy of cancer has focused on the use of monoclonal antibodies to bind to, and inhibit the activity of, inhibitory receptors on T cells such as PD-1 and CTLA-4. Antibodies have also been developed against a protein, PD-L1, that is highly expressed by cancer cells and acts as an activating ligand for PD-1. Combinations of these antibodies have proven to be very effective in treating certain types of cancer. The antibodies are injected intravenously and cause significant toxic side-effects. Although the peptides of this invention interact with different inhibitory receptors, the overall result may be the same but simply be achieved through different mechanisms.

### SUMMARY OF THE INVENTION

The present invention is directed to a method of stimulating the immune system of an immunocompromised subject, the method comprising: administering to the immunocompromised subject a composition comprising a therapeutic peptide or a multivalent structured polypeptide comprising multiple copies of the therapeutic peptide, the therapeutic peptide consisting of 5 to 8 amino acids and selected from the group consisting of:

(SEQ ID NO: 1)

VGGGS
and
X1-P-S-X2-X3-X4-X5-X6,

wherein

X1 is selected from the group consisting of H and N, or is absent;

X2 is selected from the group consisting of L, S, N, and H;

X3 is selected from the group consisting of N, K, G, L, P, and A;

X4 is selected from the group consisting of A, S, and L, or is absent;

X5 is selected from the group consisting of S and L, or is absent; and

X6 is G, or is absent;

wherein the therapeutic peptide or multivalent structured polypeptide is in an amount sufficient to activate and/or stimulate proliferation of immune cells in the subject.

In certain aspects, the therapeutic peptide or multivalent structured polypeptide stimulates proliferation of immune cells selected from the group consisting of macrophages; dendritic cells; natural killer cells; natural killer T cells; CD3+, CD4+ and CD8+ T cells; B cells; and combinations thereof. In one embodiment, the immune cells are memory B cells.

In some embodiments, the present invention further comprises determining (a) the level of immune cells in the immunocompromised subject before administering the composition; and (b) the level of immune cells in the immunocompromised subject after administering the composition. In some aspects, the levels of (a) and (b) are determined with flow cytometry. In other aspects, the ratio of (b) to (a) is at least 2, at least 3, at least 4, or at least 5. In yet other aspects, the ratio of (b) to (a) is at least 1.25, at least 1.5, at least 2, at least 2.5, at least 3, at least 3.5, at least 4, at least 4.5, or at least 5.

In some embodiments, the therapeutic peptide or multivalent structured polypeptide activates the immune cells by binding to: i) an activating receptor NKG2D; and/or ii) an inhibitory receptor sialic acid-binding immunoglobulin-like lectin (siglec).

In yet other embodiments, the therapeutic peptide is selected from the group consisting of: VGGGS (SEQ ID NO:1), PSSNA (SEQ ID NO:2), HPSLK (SEQ ID NO:3), NPSHPLSG (SEQ ID NO:7), and NPSHPSLG (SEQ ID NO:8).

In certain aspects, the multivalent structured polypeptide is branched. In other aspects, the therapeutic peptide functionally mimics a terminal sequence 5-acetylneuraminic acid-galactose or N-acetylgalactosamine on complex glycans, the terminal sequence being linked α(2-3) or α(2-6).

In another embodiment, the present invention is directed to a method of immunotherapy comprising: administering to a subject with cancer and/or a viral infection a composition comprising a therapeutic peptide or a multivalent structured polypeptide comprising multiple copies of the therapeutic peptide, the therapeutic peptide consisting of 5 to 8 amino acids and selected from the group consisting of:

(SEQ ID NO: 1)

VGGGS
and
X1-P-S-X2-X3-X4-X5-X6,

wherein

X1 is selected from the group consisting of H and N, or is absent;

X2 is selected from the group consisting of L, S, N, and H;

X3 is selected from the group consisting of N, K, G, L, P, and A;

X4 is selected from the group consisting of A, S, and L, or is absent;

X5 is selected from the group consisting of S and L, or is absent; and

X6 is G, or is absent;

wherein the therapeutic peptide or multivalent structured polypeptide is in an amount sufficient to activate and/or stimulate proliferation of immune cells in the subject.

In some embodiments, the subject suffers from an HIV infection. In other embodiments, the subject suffers from cancer.

The present invention is directed to compositions and methods of modulating the innate and adaptive immune systems in a subject, preferably including activation of B cells, natural killer (NK) cells, dendritic cells (DCs), T cells, and/or CD8+ cytotoxic T lymphocytes. The method comprises: administering to the subject a composition comprising a therapeutic peptide or a multivalent structured polypeptide comprising multiple copies of the therapeutic peptide, the therapeutic peptide consisting of 5 to 8 amino acids, the peptide being selected from the group consisting of:

(SEQ ID NO: 1)

VGGGS
and
X1-P-S-X2-X3-X4-X5-X6,

wherein

X1 is selected from the group consisting of H and N, or is absent;

X2 is selected from the group consisting of L, S, N, and H;

X3 is selected from the group consisting of N, K, G, L, P, and A;

X4 is selected from the group consisting of A, S, and L, or is absent;

X5 is selected from the group consisting of S and L, or is absent; and

X6 is G, or is absent;

wherein the therapeutic peptide or multivalent structured polypeptide is in an amount sufficient to increase activity and proliferation of macrophages, B cells, DCs, NK cells, natural killer T cells, T cells, and/or CD8+ cytotoxic T lymphocytes in the subject.

In one embodiment, the method preferably, further comprises determining the level of macrophages, B cells, T cells, DCs, NK cells and/or CD8+ cytotoxic T cells in the subject's blood. In this embodiment, it is advantageous to further establish a ratio of NK cells and/or CD8+ cytotoxic T cells compared to monocytes in the subject's blood.

The method also preferably further comprises administering at least one agent selected from the group consisting of: a B cell proliferation agent, cytotoxic T cell proliferation agent, and a NK cell proliferation agent,

The therapeutic peptide used is preferably selected from the group consisting of: VGGGS (SEQ ID NO:1), PSSNA (SEQ ID NO:2), HPSLK (SEQ ID NO:3), NPSHPLSG (SEQ ID NO:7), and NPSHPSLG (SEQ ID NO:8). A branched multivalent structured polypeptide comprising multiple copies of the therapeutic peptide is preferred.

The composition advantageously comprises the therapeutic peptide in an amount sufficient to induce activation of NK cells in the subject and the subject is a human. The therapeutic peptide preferably functionally mimics a terminal sequence 5-acetylneuraminic acid-galactose on complex glycans, the terminal sequence being linked α(2-3) or α(2-6).

The therapeutic peptides are advantageously configured to bind to the receptor NKG2D and/or siglecs and function as modulators of the immune system by binding to receptors on B cells, DCs, NK cells, T cells, cytotoxic T cells and/or phagocytic cells.

In one embodiment, the therapeutic peptide or multivalent structured polypeptide is administered in an amount sufficient to induce antibody-mediated cellular cytotoxicity in the subject, preferably to increase the expression of at least one endogenous cytokine from lymphocytes elected from the group consisting of: IL-2, IL-4, IL-16, IL-17, IL-21, TNF-β, IFN-γ and RANTES and/or decreases at least one endogenous cytokines elected from the group consisting of: IL-1α, IL-1β, IL-13, IL-12p40, and IL-12p70, TNF-α.

The method may advantageously further comprise the step of administering an antibody preparation admixed in an amount sufficient to enhance antibody-mediated cellular cytotoxicity. The present invention is also directed to a therapeutic composition. The therapeutic composition preferably comprises a carrier; at least one agent selected from the group consisting of: a B cell proliferative agent, a cytotoxic T cell proliferation agent, or a NK cell proliferation agent; and a therapeutic peptide or a multivalent structured polypeptide as described above. In certain embodiments, the composition further comprises an antibody preparation admixed in an amount sufficient to enhance antibody-mediated cellular cytotoxicity in a subject; or further comprises an immunoglobulin admixed with the polypeptide composition in an amount sufficient to enhance passive immunoprotection.

### BRIEF DESCRIPTION OF THE DRAWINGS

FIG. 1 illustrates a model of monovalent SVH1C (SEQ ID NO:7, space-filled structure) docked in the glycan-binding site of the lectin MAA from Maackia amurensis (accession no. 1DBN). The predicted binding energy for NPSHPLSG (SEQ ID NO:7), ΔG′=−46 kJ/mol, suggests strong interaction;

FIG. 2 illustrates a model of monovalent SVH1C (SEQ ID NO:7, space-filled structure) docked in the ligand binding site of the receptor NKG2D (accession no. 1MPU). The predicted binding energy for NPSHPLSG (SEQ ID NO:7) was ΔG′=−40 kJ/mol;

FIG. 3 illustrates a model of the final design of the peptide. Four identical, active sequences (e.g., arms 1 and 2) were extended from a central core (4), composed of tri-lysineamide, by a linker sequence (3);

FIG. 4 demonstrates and shows the structure of SVH1C ([NPSHPLSGGGGS]4K3-NH2). N, asparagine; P, proline; S, serine; H, histidine; L, leucine; G, glycine. The molecular weight of the peptide is 4,593.9. A linker sequence (GGGS) extends the active sequence from the tri-lysine core.

FIG. 5 shows the binding of mono-, bi- and quadravalent SV6B (SEQ ID NO:3) to monosaccharide-specific lectins. Binding of the quadravalent peptide was set as 100%. The assay contained 25 pmoles of the quadravalent peptide, 50 pmoles of the bivalent peptide, and 100 pmoles of the monovalent peptide to provide an equal number of peptide sequences per well. The peptides contained a biotin tag at the C-terminus, which anchored the peptides in wells of microtiter plates coated with streptavidin [published in ref. 16]. The data points are the mean±SD from measurements with seven different lectins.

FIG. 6 shows binding of quadravalent SV6B (SEQ ID NO:3), SVH1D (SEQ ID NO:8) and SVH1C (SEQ ID NO:7) to lectins. In order, left to right, the lectins were Helix pomatia; Griffonia simplicifolia; Triticus vulgaris (wheat germ agglutinin); Dolichos biflorus; Ulex europaeus; Sambucus nigra (SNA1) and Maackia amurensis (MAA) [published in ref. 16];

FIG. 7 shows a comparison of binding of SVH1C (SEQ ID NO:7) to MAA (circles) and SNA1 (squares) as a function of the amount of peptide added to wells containing bound lectin [16].

FIG. 8 shows a comparison of inhibition of binding of SVH1C (SEQ ID NO:7) to MAA (squares) and SNA1 (circles) by fetuin. Peroxidase-conjugated lectins were added to wells along with the indicated amount of fetuin to 100 pmoles of peptides bound to streptavidin. Lectin bound in the absence of fetuin was assigned a value of 100% [16].

FIG. 9 illustrates the binding of SVH1C (SEQ ID NO:7) to the receptor NKG2D, assayed by isothermal microcalorimetry. Increments of SVH1C (100 μM) were injected into a reaction chamber containing 11 μM (monomer) NKG2D.

FIG. 10 illustrates the binding of SVH1C (SEQ ID NO:7) to lectin-type receptors, siglecs (sialic acid-binding Ig-like lectin receptors) and other lectin-type receptors in a solid-phase assay. The buffer in these assays was PBS containing 0.05% Tween-20. The Figure shows the amount of streptavidin-peroxidase bound to SVH1C that was bound to the receptors. The receptors were Fc-chimeras and were assayed in protein NG-coated microtiter wells. Siglec-1 and CLEC10a contained a C-terminal His tag and were assayed in a separate experiment with Ni-coated wells. SEM was determined from four independent experiments run in duplicate. Inhibition by fetuin is shown by the average of two assays in which the glycoprotein was added at 10 μM (second, black bar) or 30 μM (third, light grey bar) in each receptor group. Binding was measured by a colorimetric assay of peroxidase.

FIG. 11 illustrates the binding of SVH1C (SEQ ID NO:7) to receptor NKG2D in a solid-phase assay. A, The amount of streptavidin-peroxidase bound to SVH1C that was bound to the receptor was measured after extensive washing with PBS containing 0.05% Tween-20. Fetuin (5 μM, black bar; 10 μM, light grey bar; 30 μM, grey bar) and sialyllactose (12 μM, black bar; 20 μM, light grey bar; 40 μM, grey bar) were included as inhibitors. Binding was measured by a colorimetric assay of peroxidase. This assay was performed three times. B, Graphical representation of inhibition of binding of SVH1C (SEQ ID NO:7) to NKG2D by fetuin (circles) or sialyllactose (squares).

FIG. 12 illustrates the changes in phosphorylation of cell-surface immunoreceptors after treatment of human PBMCs with 100 nM SVH1C (SEQ ID NO:7) for 5 min. Cell lysates were spread on an array of capture antibodies and phosphorylated forms were detected by a phospho-tyrosine antibody (R&D Systems, Minneapolis, Minn.). The phosphorylated forms of CD229, FcγRIIA, LAIR-1, Siglec-2, -3, -5, -7, -9, and -10 decreased, while BLAME increased. FcRH4 and SHP-1 did not change significantly. This experiment showed that the peptide has dramatic effects on human cells.

FIG. 13 illustrates that alternate-day subcutaneous injection of SVH1C (SEQ ID NO:7) into C57BL/6 mice resulted in relatively large increases in populations of immune cells in the peritoneal cavity. The bars, in increasing darkness, show populations of specific cell types at 1, 3 and 5 days of treatment, i.e., 24 hours after each injection at day 0, 2 and 4. Peritoneal cells were obtained from 3 animals, pooled, and analyzed by flow cytometry. The markers used to identify cell types are listed across the top of the figure. The total number of each cell type is plotted, with the scale indicated at the top of each cell type. Cells are identified by the usual designations across the bottom of the figure. An asterisk indicates the activated populations that express CD69.

FIG. 14 shows inhibition of HIV-1 replication in PBMC cultures by SVH1C (SEQ ID NO:7) and SV6B (SEQ ID NO:3) without anti-HIV antibodies.

### DETAILED DESCRIPTION OF THE PREFERRED EMBODIMENTS

The present invention is directed to compositions and methods for activation and proliferation of B cells, DCs, NK cells, T cells and/or CD8+ cytotoxic T lymphocytes.

### Peptidic Mimetics of Glycan Ligands of Receptors

An important component of immune system stimulation by the peptides is activation and proliferation of B cells, DCs, NK cells, T cells and CTL (cytotoxic T lymphocytes) in addition to activation of phagocytic cells. To this end, peptidic mimetics of the glycan 5-acetyl-neuraminic acid-galactose [Neu5Ac(α2-3)Gal and Neu5Ac(α2-6)Gal] were designed. These glycans bind to NKG2D, an important activating receptor on NK cells, γδ T cells and CD8⁺ cytotoxic T cells [12, 15], and to the family of siglecs (sialic acid-binding Ig-like lectin) receptors that is present on most cells of the immune system and are generally inhibitory receptors [12]. Whereas identified endogenous ligands of NKG2D are several protein-based activating ligands [10, 17], binding of glycans should also activate these cells [13, 14]. Activation of phagocytes occurs by binding of peptides to siglecs or other receptors on these cells. The therapeutic peptides consist of a multivalent structure in which the arms consist of sequences only 9 to 12 amino acids long (including a linker sequence). The active sequences of the relevant peptides that were described previously [U.S. Pat. No. 7,811,995 incorporated by reference thereto] are VGGGS (SEQ ID NO:1), HPLSK (SEQ ID NO:3), NPSHPLSG (SEQ ID NO:7) and NPSHPSLG (SEQ ID NO:8). Preferably, the peptides are in substantially pure form. Typically it is desired that the peptides be at least 70%, more preferably at least 80%, and most preferably at least 95% pure by weight. In one embodiment the N-terminus may also be acetylated.

In a preferred embodiment, the peptides of the invention comprise a peptide construct with at least two arms. The construct typically has a central framework and each arm comprises a therapeutic sequence linked to the central framework via a linker. Each therapeutic sequence of the peptide construct can be the same or different. In a preferred embodiment, the therapeutic sequence is the same for each arm of peptide construct. The therapeutic sequence is preferably selected from the group of therapeutic peptides described above. The present invention also provides therapeutic compositions comprising at least one peptide of the invention and a pharmaceutically acceptable carrier. In a preferred embodiment, the composition is an immunostimulatory composition, preferably further comprising an antigen and/or an antibody preparation admixed therewith in an amount sufficient to enhance antibody-mediated cytotoxicity or phagocytosis. Alternatively, the composition may comprise an immunoglobulin admixed with the therapeutic peptide in an amount sufficient to substantially enhance passive immune protection, e.g., at least 30% increase compared to the control.

In another embodiment, the therapeutic compositions comprises a carrier; at least one agent selected from the group consisting of: a B cell proliferation agent, a dendritic cell proliferation agent, a cytotoxic T cell proliferation agent, or a NK cell proliferation agent; and a therapeutic peptide or a multivalent structured polypeptide as described above. In certain embodiments, the composition further comprises an antibody preparation admixed in an amount sufficient to enhance antibody mediated cellular cytotoxicity in a subject; or further comprises an immunoglobulin admixed with the polypeptide composition in an amount sufficient to enhance passive immunoprotection.

Preferred cytotoxic T cell proliferation agents and/or NK cell proliferation agents include molecules that increase IL-2, IL-15 and IL-21 expression. Alternatively, molecules that induce IL-12 and IL-18 expression are included.

The peptides of the invention are useful in treating the subject having a disease, especially those diseases treatable by endogenous induction of antibodies against invading pathogens or endogenous antigens of harmful cells. The peptides of the invention can specifically be used to treat such diseases as viral infections, cancer, bacterial and yeast infections, and/or other autoimmune diseases which require treatment through stimulation of the immune system. Such autoimmune diseases include rheumatoid arthritis, psoriasis; dermatitis; systemic scleroderma and sclerosis; responses associated with inflammatory bowel disease; Crohn's disease; ulcerative colitis; respiratory distress syndrome; adult respiratory distress syndrome (ARDS); dermatitis; meningitis; encephalitis; uveitis; colitis; glomerulonephritis; allergic conditions; eczema; asthma; conditions involving infiltration of T cells and chronic inflammatory responses; atherosclerosis; leukocyte adhesion deficiency; systemic lupus erythematosus (SLE); diabetes mellitus; multiple sclerosis; Reynaud's syndrome; autoimmune thyroiditis; allergic encephalomyelitis; Sjorgen's syndrome; juvenile onset diabetes; immune responses associated with acute and delayed hypersensitivity mediated by cytokines and T lymphocytes; tuberculosis; sarcoidosis; polymyositis; granulomatosis; vasculitis; pernicious anemia (Addison's disease); diseases involving leukocyte diapedesis; central nervous system (CNS) inflammatory disorder; multiple organ injury syndrome; hemolytic anemia; myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; idiopathic thrombocytopenic purpura (ITP) and autoimmune thrombocytopenia.

The invention encompasses methods of substantially activating subsets of lymphocytes in a subject, in particular NK cells that attack diseased cells directly or by antibody-dependent cellular cytotoxicity, which complements activation of Fc-mediated phagocytosis, to treat a subject. In a preferred embodiment, HIV-1 replication is inhibited in the subject by at least 50%, more preferably by at least 90% as compared to a control and/or levels prior to administration of the peptide in the subject. In the presence of antibodies, inhibition may reach 100%.

In a preferred embodiment, to provide a non-specific therapeutic agent with a relatively broad front, an agent that activates DCs, B cells, T cells, NK and cytotoxic T cells preferably works in concert with the phagocytic cells of the immune system. The peptides of the present invention can accomplish this goal by concomitantly stimulating the immune cells, including NK cells and phagocytes, and to respond in particular to the presence of pathogen-directed antibodies. Treatment with the peptides of the present invention therefore preferably induce activation of cells of the immune system in vivo and provide a sustained endogenous defense against the pathogen.

In the following description, and for the purposes of explanation, numerous specific details are set forth in order to provide a thorough understanding of the various aspects of the invention. It will be understood, however, by those skilled in the art, that the structures, compositions, and methods are sometimes shown or discussed generally in order to avoid obscuring the invention. In many cases, a description of the material and operation is sufficient to enable one to implement the various forms of the invention. It should be noted that there are many different and alternative technologies and treatments to which the disclosed inventions may be applied, and the full scope of the inventions is not limited to the examples that are described below. Unless otherwise defined, all technical and scientific terms used herein have the same meaning as commonly understood by one of ordinary skill in the art to which this invention belongs.

The term “pharmaceutically acceptable” as used herein means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which an active ingredient is administered. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. In addition, other excipients can be used.

Preferably, the subject being treated by the methods described herein is a mammal, e.g., monkey, dog, cat, horse, cow, sheep, pig, and more preferably the subject is human.

“Effective amount” or “therapeutically effective amount” is meant to describe an amount of therapeutic peptide or composition of the present invention effective to modulate the innate and adaptive immune systems and/or treat or prevent a disease in a subject and thus produce the desired therapeutic effect in the subject.

Typical compositions and dosage forms may comprise one or more excipients. Suitable excipients are well known to those skilled in the art of pharmacy, and non-limiting examples of suitable excipients are provided herein. See, e.g., Remington's Pharmaceutical Sciences, 18th ed., Mack Publishing, Easton Pa. (1990).

The present invention comprises therapeutic peptides, compositions of those therapeutic peptides for administration to a subject in need, and methods to stimulate the immune system of a subject through the administration of compositions containing those therapeutic peptides. In general, the advantage of this invention is the modulated release of specific cytokines and the stimulation of immune cells, including but not limited to B cells, NK cells, CD8⁺ T cells and phagocytes, to respond to the presence of pathogen-directed antibodies. Non-limiting examples of cytokines include immunoregulatory proteins such as interleukins and interferons, which are secreted by cells of the immune system and can affect the immune response. A non-limiting example of the stimulation of immune cells is the induction of Fc-mediated phagocytosis. An additional example is direct activation of NK cells for antibody-dependent cellular cytotoxicity. A further example is activation of NK cells and CTL to lyse infected or cancer cells by direct cellular cytotoxicity.

The single letter designation for amino acids is used predominately herein. As is well known by one of skill in the art, the single letter designations are as follows: A is alanine; C is cysteine; D is aspartic acid; E is glutamic acid; F is phenylalanine; G is glycine; H is histidine; I is isoleucine; K is lysine; L is leucine; M is methionine; N is asparagine; P is proline; Q is glutamine; R is arginine; S is serine; T is threonine; V is valine; W is tryptophan; Y is tyrosine.

The therapeutic peptide is preferably 5 to 8 amino acids. Preferred therapeutic peptides are selected from the group consisting of:

(SEQ ID NO: 1)

VGGGS
and
X1-P-S-X2-X3-X4-X5-X6,

wherein

X1 is selected from the group consisting of H and N, or is absent;

X2 is selected from the group consisting of L, S, N, and H;

X3 is selected from the group consisting of N, K, G, L, P, and A;

X4 is selected from the group consisting of A, S, and L, or is absent;

X5 is selected from the group consisting of S and L, or is absent; and

X6 is G, or is absent.

In a most preferred embodiment, the therapeutic peptide is selected from the group consisting of: VGGGS (SEQ ID NO:1), PSSNA (SEQ ID NO:2), HPSLK (SEQ ID NO:3), NPSHPLSG (SEQ ID NO:7), and NPSHPSLG (SEQ ID NO:8).

Multivalent structured polypeptides comprising multiple copies of the therapeutic peptide are preferred. In one embodiment, the multivalent structured polypeptide comprises a construct and at least two arms, the construct having a central framework and each arm comprising a therapeutic peptide sequence linked to the central framework via a linker, wherein each therapeutic sequence is preferably the same.

As used herein, “construct” is defined as the entire molecule and comprises the central framework linked with at least two arms. In a preferred embodiment, the construct comprises the central framework linked to 2 or more arms, e.g., 3, 4, 5, 6, 7, 8, 9, 10, 11, or 12 arms, preferably 2 to 8 arms. In a further preferred embodiment, the construct comprises the central framework linked to 4 arms. Each arm within the construct may consist of the same or different therapeutic sequence and/or linker. In one preferred embodiment, the therapeutic sequence is the same between arms.

The “central framework” provides a structure for attaching the arms. The central framework is based on a core molecule which has at least two functional groups to which molecular branches having terminal functional groups are bonded, e.g., a tri-lysine to which the peptide arms are added. Such molecules may be developed or created to present a varying number of branches, depending on the number of monomers branched from the core molecule. Each terminal functional group on each branch provides a means of attachment to an arm. Non-limiting examples of preferred central framework include: ethylenediamine (1,2-ethanediamine), ethylene glycol (1,2-dihydroxyethane), polyols such as glycerol, 3,5-diaminobenzoic acid, 1,3,5-triaminobenzene, and monocarboxylic-diamino compounds of intermediate length. Preferably, the monocarboxylic-diamino compounds are within the range of 2 to 10 carbons in length. Non-limiting examples of such compounds are 2,3-diaminopropionic acid and 2,6-diaminocaproic acid. In a more preferred embodiment, the monocarboxylic-diamino compound is 6 carbons in length. Compounds that provide an aromatic central framework which absorbs light may be beneficial for determining peptide concentration as well. The carboxyl group of the monocarboxylic-diamino compounds allows the addition of C-terminal tags including biotin derivatives. In a preferred embodiment, the central framework comprises a tri-lysine core (a lysine residue as the central molecule bonded to two lysine residues, each through its carboxyl group, to one of the amino groups of the central lysine residue), providing a central framework for four arms.

The “arm” comprises the therapeutic sequence, plus the linker. The “linker” comprises a peptide chain or other molecule that connects the central framework to the core sequence. In a preferred embodiment, the linker comprises, but is not limited to, certain linker peptide sequences, polyethylene glycol, 6-aminocaproic acid (6-aminohexanoic acid), 8-aminooctanoic acid, and dextran. In a most preferred embodiment, the linker is GGGS (SEQ ID NO:9), GGGSGGGS (SEQ ID NO:10), SSSS (SEQ ID NO:11), SSSSSSSS (SEQ ID NO:12), or variations thereof. The length of the linker can be adjusted, for example, the linker GGGS (SEQ ID NO:9) can be repeated to provide variable lengths, e.g., repeated twice (GGGSGGGS (SEQ ID NO:10)), or even three or more times; additional serine residues could be added to SSSS (SEQ ID NO:11) to also produce varying lengths of the linker. The therapeutic peptide preferably functionally mimics a terminal sequence 5-acetylneuraminic acid-galactose on complex glycans, the terminal sequence being linked α(2-3) or α(2-6). In some aspects, the therapeutic peptide functionally mimics a terminal sequence 5-acetylneuraminic acid-galactose or N-acetylgalactosamine on complex glycans, the terminal sequence being linked α(2-3) or α(2-6). The therapeutic peptides are advantageously configured to bind to the receptor NKG2D and/or sialic acid-binding immunoglobulin-like lectins and function as modulators of the immune system by binding to receptors on B cells, DCs, NK cells, T cells, cytotoxic T cells and/or phagocytic cells.

The therapeutic peptide is preferably administered in an amount sufficient to induce activation of NK cells in the subject and the subject is a human. In one embodiment, the therapeutic peptide or multivalent structured polypeptide is administered in an amount sufficient to induce antibody-mediated cellular cytotoxicity in the subject, preferably to increase the expression of at least one endogenous cytokine from lymphocytes elected from the group consisting of: IL-2, IL-4, IL-16, IL-17, IL-21, TNF-β, IFN-γ and RANTES and/or decreases at least one endogenous cytokines elected from the group consisting of: IL-1α, IL-β, IL-13, IL-12p40, and IL-12p70, TNF-α.

The method may advantageously further comprise the step of administering an antibody preparation admixed in an amount sufficient to enhance antibody-mediated cellular cytotoxicity.

The step of determining the level of immune cells such as B cells, NK cells and/or CD8⁺ cytotoxic T cells in the subject's blood is done using well known methods in the art, e.g., flow cytometric analysis of peripheral blood mononuclear cells with use of antibodies against cell-specific surface markers. It is advantageous to further establish a ratio of NK cells and/or CD8+ cytotoxic T cells compared to monocytes in the subject's blood. In a preferred embodiment, the ratio of NK cells or CD8+ cytotoxic T cells to monocytes is 3:1 or more preferably 4:1. The present invention is most effective with a higher ratio NK cells and/or CD8+ cytotoxic T cells compared to monocytes.

The present invention identifies a series of peptides that stimulate immune response and modulate the release of specific cytokines. Thus, in a first aspect, the present invention provides a therapeutic peptide consisting of 9 to 12 amino acids in length (including a spacer sequence). In a preferred embodiment, the therapeutic peptide is in a substantially purified form. As used herein, the term “substantially purified” refers to material which is substantially or essentially free from components which normally accompany it as found in its synthesized state. When the material is synthesized, the material is substantially or essentially free of cellular material, gel materials, culture medium, chemical precursors, contaminating polypeptides, nucleic acids, endotoxin, and other organic chemicals. Preferably, the peptide is purified to represent greater than 90% (peptide content) of all organic molecular species present. More preferably the peptide is purified to greater than 95% (peptide content), and most preferably the peptide is purified to essential homogeneity, wherein other organic molecular species are not detected by conventional techniques. Advantageously, the therapeutic peptide is reacted with acetic anhydride to acetylate the N-terminus of the therapeutic peptide. Acetylation protects the peptide from N-terminal degradation and therefore is preferred.

### Scientific Basis of the Invention

Peptide sequences were identified by computer-aided molecular modeling of docking to the sugar-binding site of plant lectins, which served as receptor analogs. The concept underlying the design of Susavion's peptides had several components. From knowledge that a number of receptors on cells of the immune system bind carbohydrate ligands [18, 19], we focused on developing peptidic mimetics of these glycan ligands. Peptides of 5 to 8 amino acids in length fill the glycan binding site of lectins and receptors and are sufficiently short to be invisible to the antigen-presenting processes of the immune system. An important aspect of the final peptide is a multivalent structure that is capable of cross-linking receptors, an event that is critical to initiation of a signal transduction pathway within the cell [20, 21]. To determine the most effective amino acid sequence of a peptide, molecular modeling was performed of docking of a single (monovalent) sequence into the glycan-binding site of well-characterized plant lectins, which were selected as analogs of cell-surface receptors. The crystal structures of the lectins were downloaded from the Protein Data Bank (PDB). ArgusLab 4.0.1 software (Mark A. Thompson, Planaria Software LLC, Seattle, Wash.) was used for modeling. Amino acid residues that comprise the binding site of a lectin or receptor were selected from the literature that describes each protein. Through this approach, unique peptide sequences were evaluated by predicted binding energy. These in silico experiments predicted that some peptides would bind to a variety of lectins with sufficiently high affinities to encourage further characterization.

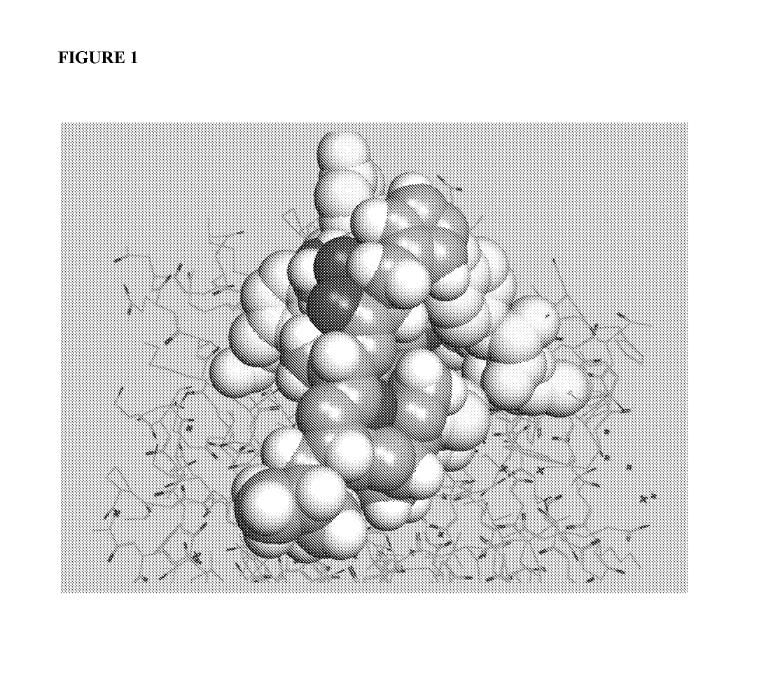

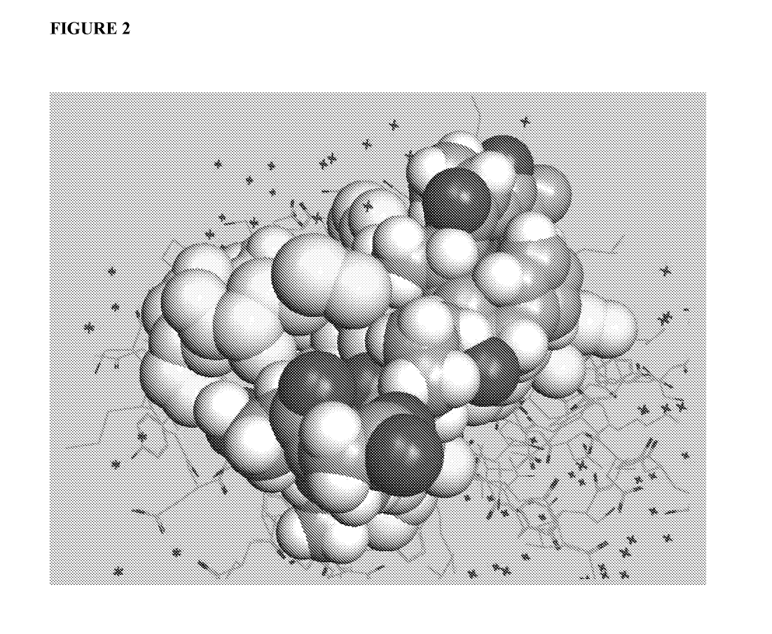

A model for interaction of the peptide designated SVH1C (SEQ ID NO:7) with the glycan-binding site of the lectin MAA from Maackia amurensis (accession no. 1 DBN), which binds with high specificity to trisaccharides containing a terminal Neu5Ac(α2-3)Gal linkage, is shown in FIG. 1. The predicted value for ΔG′ of −46 kJ/mol corresponds to a KD of 1×10⁻⁸ M for the monovalent peptide. Cell-surface receptors that bind to these sugars include the family of siglecs and NKG2D, an important activating receptor on NK cells and CD8⁺ cytotoxic T cells. Although NKG2D has a variety of peptide/protein ligands in vivo [10, 17], the C-type lectin domain of this receptor suggested that it may also bind to glycans. This hypothesis was affirmed when Imaizumi et al. [15] demonstrated that NKG2D binds glycans with the same specificity as the lectin MAA. The ligand binding site of NKG2D (accession no. 1MPU) was constructed from data presented by Li et al. [22] and McFarland et al. [23]. Modeling predicted highly favorable binding energy to NKG2D, with a ΔG′ of −40 kJ/mol, which corresponds with a K_{D} of about 1×10⁻⁷ M (FIG. 2).

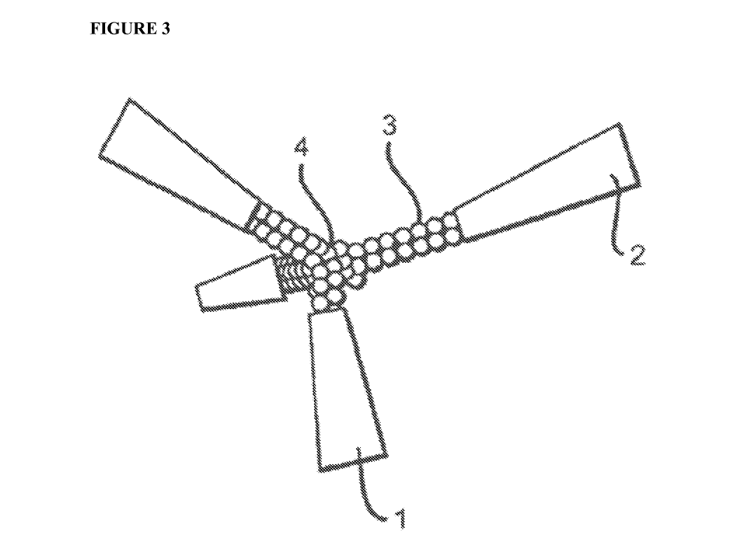

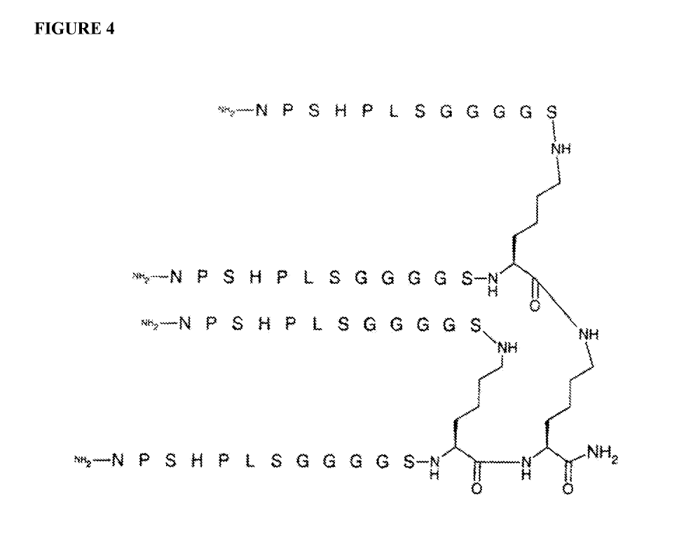

The short peptide sequence was then incorporated into multivalent structures (FIG. 3). This design was based on the concept of avidity as a function of ligand density and entropic factors. The theoretical basis for Multivalency was provided by Mammen et al. [24], Dimick et al. [25] and Cairo et al. [26]. Multivalency should provide much more favorable binding energy than predicted by molecular modeling. Although monovalent peptides should be active, multivalency of ligands provides high avidity interactions and facilitates cross-linking of receptors, which is often required for activation of cellular responses [20, 21]. The final quadravalent structure with the active sequence NPSHPLSG (FIG. 4) was selected as the lead peptide.

### Direct Binding of Peptides to Lectins

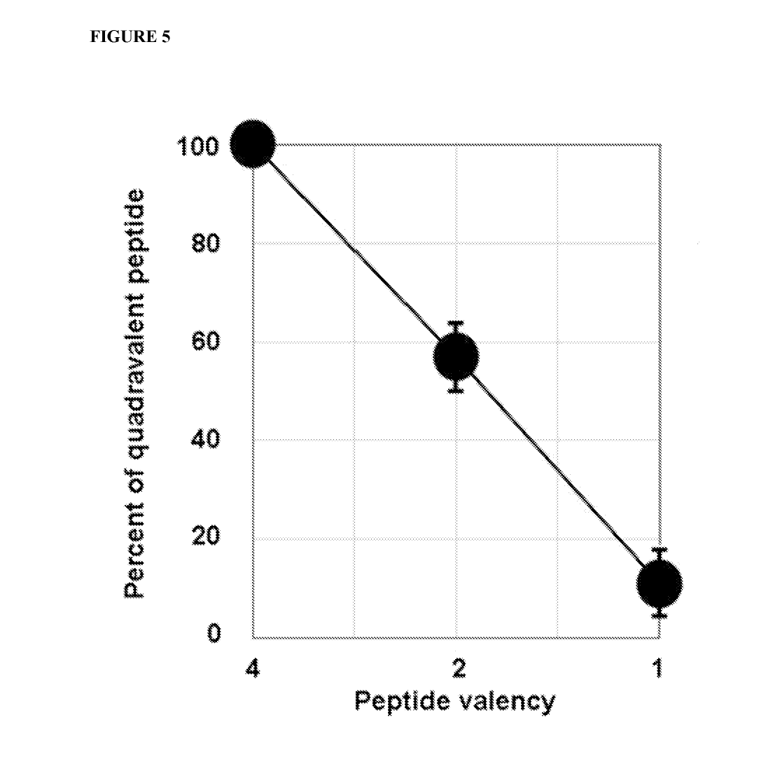

The concept of the importance of valency for high affinity binding [24-26] was tested directly by synthesizing a monovalent peptide in which the sequence was extended from the α-amino group of ε-biotinyl-lysine amide. The bivalent molecule contained the peptide sequence extended from the α and ε amino groups of a second lysine residue linked to ε-biotinyl-lysine amide. The quadravalent molecule contained the peptide sequence extended from the four amino groups of a tri-lysine scaffold in which the C-terminal amide group (as in FIG. 4) was replaced with ε-biotinyl-lysine amide. Binding of the peptides to lectins was performed with a solid-phase assay in which the C-terminal ε-biotinyl-lysine amide anchored the peptide to streptavidin that was bound in microtiter plate wells. This arrangement should allow maximal flexibility of the N-terminal sequences for interaction with lectins. After the peptide and lectins were incubated for 1 h, the wells of microtiter plates were washed extensively, which should retain only strongly bound lectin. The extent of binding was measured with a colorimetric assay for peroxidase conjugated to the lectins. To achieve equal numbers of sequences, 25 pmoles of the quadravalent peptide, 50 pmoles of the bivalent peptide, and 100 pmoles of the monovalent peptide were added per well (streptavidin binding capacity, 125 pmoles biotin). As presented in FIG. 5, in a test of this concept with seven different lectins, a quadravalent peptide bound to lectins with approximately 10-fold higher avidity than the monovalent structure with the same sequence. The quadravalent peptide bound approximately two times more lectin than the bivalent peptide, which bound five times more than the monovalent peptide. This assay was performed with peptide SV6B (HPSLK, SEQ ID NO:3) to obtain a general pattern, because SVH1C (NPSHPLSG, SEQ ID NO:7) did not bind detectably to lectins specific for monosaccharides [16]. However, when assayed with MAA and SNA1, the pattern of binding of SVH1C to these lectins was similar to that shown in FIG. 5. Peptides without the biotin tag were not retained in the assay.

The lectins, their specificities, and PDB accession numbers were the GalNAc/Gal-specific lectin from Helix pomatia (2CE6), GalNAc-specific lectin from Vicia villosa (1N47), GalNAc-specific lectin from Dolichos biflorus (1LU2), the lectin from Triticus vulgaris (1WGT), which binds GlcNAc and Neu5Ac but also clusters of GalNAc, and the lectin from Canavalia ensiformis (3CNA), which binds Man. The lectins MAA and SNA1, described below, were also included in the study.

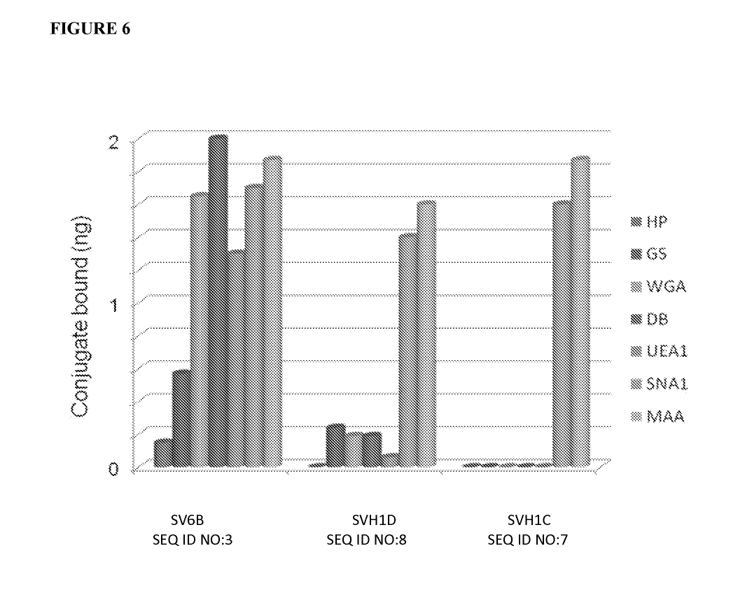

As shown in FIG. 6, SVH1C did not bind detectably to lectins specific for monosaccharides. However, strong binding was found with the lectin MAA from Maackia amurensis and a related lectin, SNA1, from Sambucus nigra, which are specific for complex glycans. Whereas MAA requires three intact terminal sugars with the sequence Neu5Ac(α2-3)Gal(β1-4)GlcNAc/Glc- [27], SNA1 requires a terminal disaccharide with the structure Neu5Ac(α2-6)Gal/GalNAc- [28]. The binding data indicate that SVH1C was not specific for the Neu5Ac-Gal linkage.

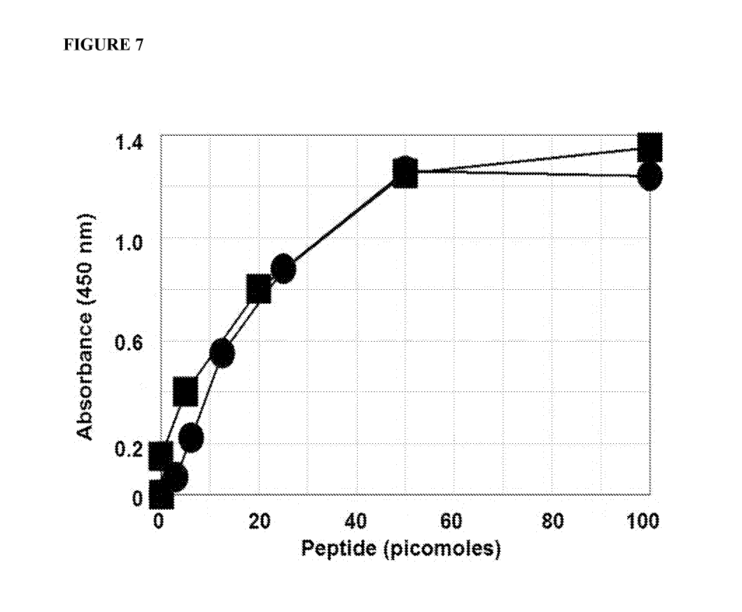

Binding of SVH1C to MAA and SNA1 was examined further as a function of the amount of peptide added to assay wells containing bound lectin. As shown in FIG. 7, binding of SVH1C to MAA and SNA1 was similarly saturated at about 100 pmoles, with half-maximal binding obtained near 25 pmoles peptide per well. From assays of additional peptides, we found that binding of one of the quadravalent peptides designated SVD2 with the sequence VSNQH (SEQ ID NO:13) to MAA and SNA1 could not be detected above blank values in subsequent experiments (data not shown). SVD2 was therefore chosen as a control (inactive) peptide in further experiments.

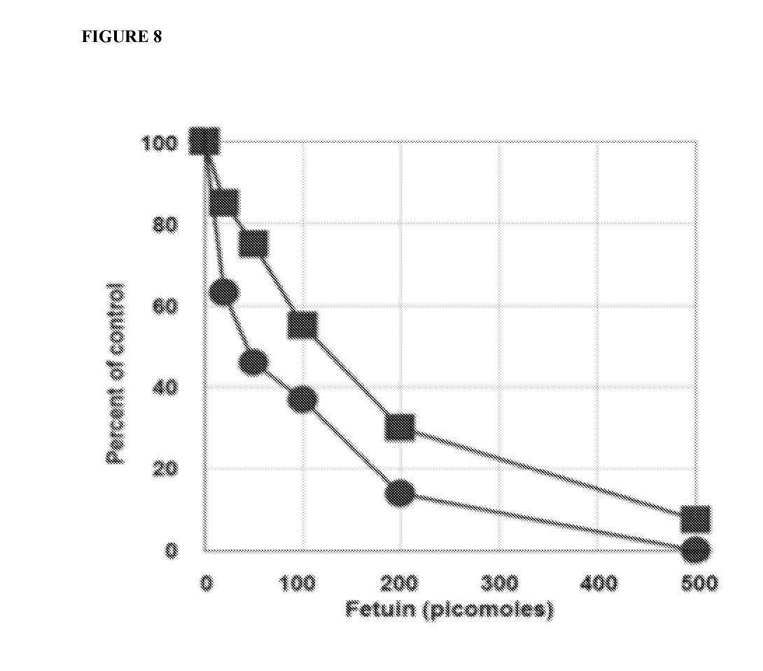

To confirm that the peptides interacted with carbohydrate-binding sites on the lectins, competition binding assays were performed with the glycoprotein fetuin. Each fetuin molecule contains collectively 12 to 15 oligosaccharides that terminate predominantly as Neu5Ac-Gal, with nearly equal α(2-3) and α(2-6) linkages, on three N-linked and three O-linked glycans [29, 30]. If indeed SVH1C mimics Neu5Ac-Gal termini, fetuin should compete effectively with the peptides and inhibit their binding to MAA and SNA1. As shown in FIG. 8 [16], essentially complete inhibition of binding of SVH1C to the lectins was achieved with 500 pmoles of fetuin per well (250 μg/mL), with about 50% inhibition at 100 pmoles (equal concentrations of peptide and fetuin). These results indicate that the peptides bind the lectins at least as strongly as a natural multivalent glycoprotein. The specificities of MAA for α(2-3) and of SNA1 for α(2-6) linkages are very strong [31], which supports the peptides as mimics of Neu5Ac-Gal with either linkage.

To further examine the ability of fetuin to inhibit binding of peptides to MAA and SNA1, concentrations were chosen that were one-third, equal or 5-fold the molar concentration of peptide in the assay. Similar to the data shown in FIG. 8 [16], fetuin inhibited binding of SVH1C to the lectins in this assay in a concentration-dependent manner, with 90% inhibition at a 5-fold excess. To confirm that fetuin inhibited binding by displacing the peptide from a glycan-binding site, the glycoprotein was digested with α-neuraminidase to remove the terminal Neu5Ac residue. Binding of SVH1C to MAA was completely restored and nearly completely restored to SNA1, even when fetuin thus depleted of the terminal sugar was added at 10-fold the molar concentration peptide. These data, reported in reference 16, indicated that SVH1C was interacting with the lectins at glycan-binding sites, and therefore with SVH1C is a strong Neu5Ac-Gal mimetic.

The binding of SVH1C to lectins such as MAA and SNA1 suggests that the peptide mimics Neu5Ac-Gal sequences on the termini of complex glycans. This sequence is a ligand for the receptor NKG2D on NK cells and by T cells and CD8+ cytotoxic T cells [15]. Also, a family of 14 lectin-type receptors, the siglecs (sialic acid-binding Ig-like lectins), binds Neu5Ac-Gal-sequences (reviewed in reference 12). The siglecs are thought to promote cell-cell interactions and regulate the functions of cells in the innate and adaptive immune systems through glycan recognition. These receptors are possible targets of the peptide, as predicted by molecular modeling (FIG. 2). Whereas NKG2D is specific for the Neu5Ac(α2-3)Gal linkage, members of the siglec family express specificity for the α(2-3) or α(2-6) linkages. Thus the peptides have the flexibility to bind to all of these receptors.

### Binding of SVH1C to NKG2D and Siglecs

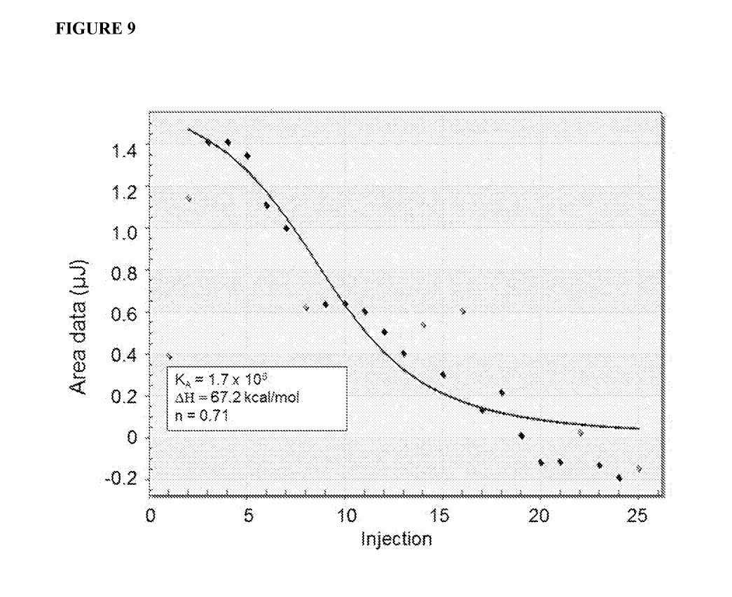

Binding of SVH1C to NKG2D was assayed by isothermal microcalorimetry. SVH1C (100 μM) was titrated into a solution of NKG2D (11 μM, monomer concentration) and changes in heat content of the system were measured. FIG. 9 shows a binding curve that yielded a binding constant, K_{A}=1.7×10⁶ M-1, which corresponds to a K_{D} of 6×10⁻⁷ M (0.6 μM). The energy of binding was similar to that predicted by molecular modeling (FIG. 2).

The thermodynamics of the binding reaction, with a positive AH, suggests that the entropy was a major factor in the strong binding. Similar characteristics of ligand binding to NKG2D, with positive enthalpy and a large entropy contribution, were reported in the literature (22, 23, 32, 33). Furthermore, NKG2D occurs as a homodimer but the concentration was introduced into the analysis as the monomeric number of binding sites. In the analysis, the stoichiometry, n, yielded a value of 0.71. This number suggests that approximately half of the bound peptide cross-linked the two binding sites of the dimeric protein or that only half of the protein had both binding sites filled.

NKG2D is not known to function as a glycan receptor in vivo, although the Neu5Ac(α2-3)Gal structure binds to the C-type lectin domain of the receptor (15). On the other hand, the siglecs have been characterized as receptors that bind Neu5Ac(α2-3) or (α2-6)Gal [12, 34]. These receptors function as either inhibitory or activating when bound with a ligand. Siglec-1 is expressed on macrophages and is involved in cellular adhesion but also enhances endocytosis [12]. As such, it enhances infection of these cells by HIV-1 by binding to glycans on the envelop of the virus [35, 36]. Expression of other siglecs is distributed on other cells of the immune system [12, 13]. Because siglecs are known to bind to the same glycans as the lectins MAA and SNA1 (see FIG. 6), it is expected that peptide SVH1C will also bind to siglecs.

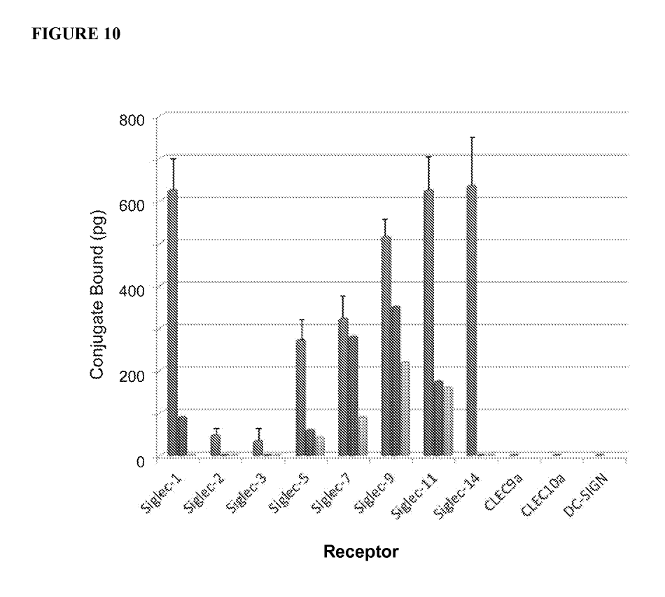

Direct binding of SVH1C to siglecs was demonstrated by a solid-phase assay in which recombinant chimeric siglecs were bound in microtiter wells coated with protein A/G. The chimeric siglecs contained an N-terminal glycan-binding domain and a C-terminal Fcγ domain, which bound strongly to protein A. Biotinylated peptides were then incubated with the siglecs, the wells were stringently washed and the bound peptide was detected by binding of streptavidin conjugated with peroxidase. FIG. 10 shows results of this assay with several siglecs and additional lectin-type receptors. SVH1C bound strongly to several siglecs but not to CLEC9a, CLEC10a or DC-SIGN. Binding of SVH1C was inhibited by the sialylated protein, fetuin, which indicated that the peptide likely bound at the glycan-binding site. In other experiments, a proteomic analysis of proteins fished from PBMCs with biotinylated SVH1C and streptavidin-agarose identified Siglec-15, an activating receptor found on myeloid cells (37), among the complex of proteins that bound to the peptide. Among myeloid cells that express Siglec-15 are macrophages and dendritic cells [13].

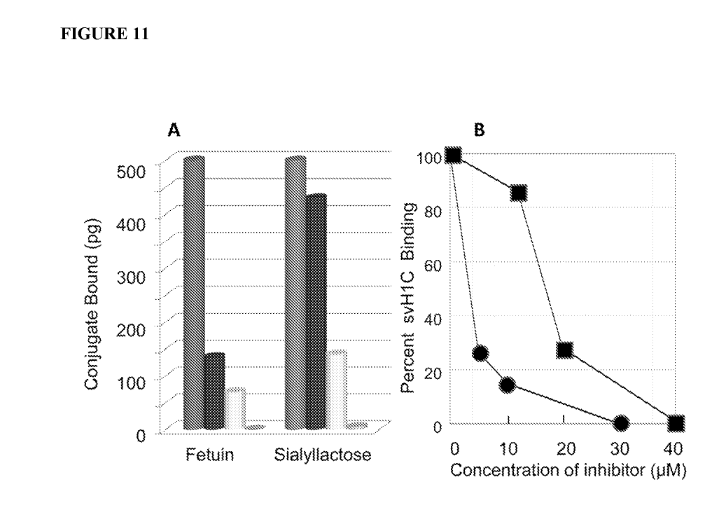

The solid-phase assay was also used to determine binding of SVH1C to NKG2D (FIG. 11). Fc-chimeric NKG2D was bound in microtiter wells coated with protein NG, which binds strongly to the Fc domain. Binding of biotinylated svH1C was measured by activity of peroxidase conjugated to streptavidin. Strong binding was observed, with a KD of approximately 1 μM. As shown in FIG. 11, binding of the peptide was inhibited to the fetuin and the trisaccharide sialyllactose, which indicated that the peptide bound in the glycan-binding site on the receptor.

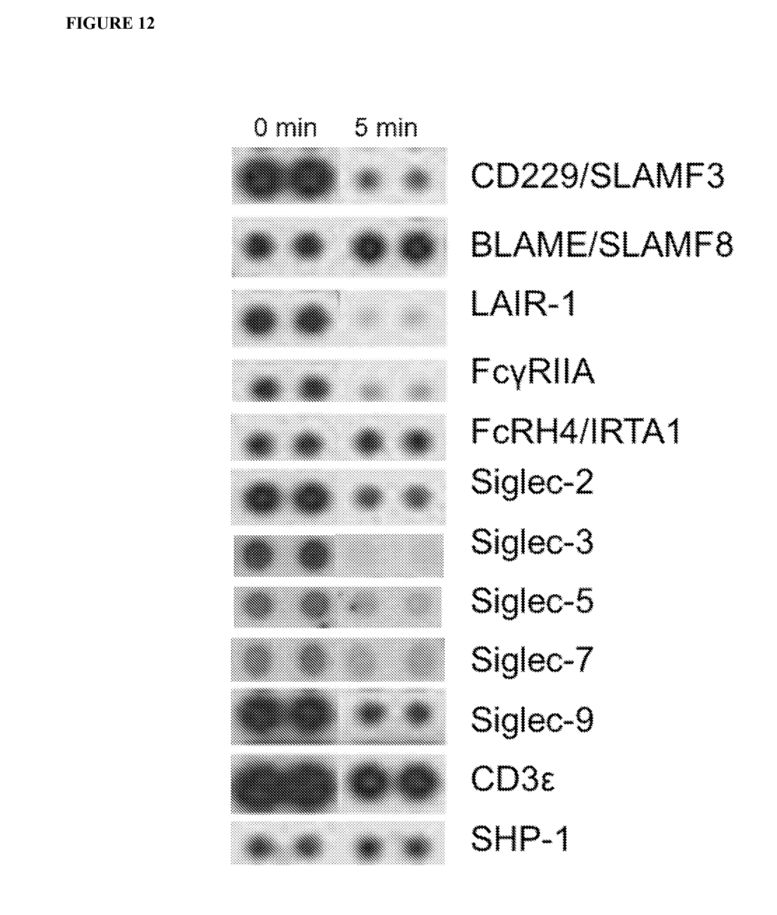

Among the siglec receptors, most are inhibitory receptors and contain an ITIM (immunoreceptor tyrosine-based inhibitory motif) within their cytosolic domain, whereas a few, in particular Siglec-14, Siglec-15, and Siglec-16 function with a cytoplasmic, activating adaptor protein, DAP12 [13, 37]. NKG2D is also an activating receptor and functions in association with the cytoplasmic, adaptor proteins DAP10 and DAP12, which contain an ITAM (immunoreceptor tyrosine-based activation motif) [10, 17]. The function of these receptors is regulated by phosphorylation of the tyrosine residue within the regulatory motif. As illustrated in FIG. 12, treatment of human peripheral blood mononuclear cells (PBMCs) with 100 nM SVH1C for 5 min caused dramatic changes in the phosphorylation state of several receptors. Phosphorylated inhibitory receptors commonly function by recruiting SHP-1, a phosphatase that inactivates other receptors [13, 37]. Thus, dephosphorylation of these receptors attenuates their activity.

### In Vivo Proliferation of Immune Cells

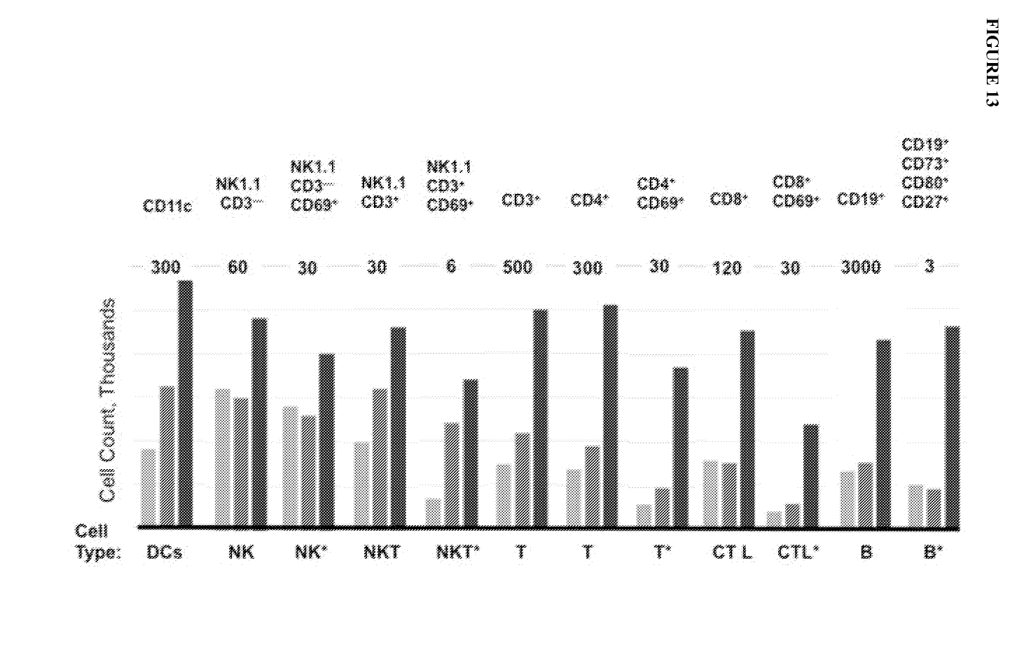

To determine whether a decrease in activity of inhibitory receptors is reflected by stimulation of proliferation of immune cells in vivo, SVH1C was injected subcutaneously every other day at a dose of 1 nanomole per gram body weight and populations of immune cells in the peritoneal cavity were measured by flow cytometry. Injections were administered on day 0, 2 and 4, and peritoneal lavage was performed to obtain immune cells. Cells from three animals at each time point were pooled and analyzed by flow cytometry. As illustrated in FIG. 13, most cells types proliferated over the period of treatment. In particular, DCs (CD11c⁺), NK cells (NK1.1⁺), CD3⁺, CD4⁺ and CD8⁺ T cells, and B cells (CD19⁺) populations increased several-fold, including those that expressed the activation marker CD69⁺. In particular, memory B cells, which express CD73, CD80 and CD273, increased significantly.

### Induction of Cytokine Release

To determine whether activation of cells by the peptides could be detected by induction of release of cytokines, cultured peripheral blood mononuclear cells (PBMCs) were treated with one peptide embodiment of the present invention and, after 4 h incubation, the medium was collected and assayed for changes in the amounts of 40 different cytokines. A therapeutic peptide construct containing four copies of the core sequence VGGGS (SEQ ID NO:1), HPSLK (SEQ ID NO:3) or NSPHPLSG (SEQ ID NO:7) was added at a concentration of 100 nM in each of the assays. Approximately 3 million cells of frozen human PBMCs were thawed at 37° C. and transferred to a 50 mL conical tube where 8 mL of wash medium were added slowly. Then an additional 8 ml of wash medium were added and swirled to mix. The cells were then centrifuged at 330 g for 10 min, the supernatant was removed and the pellet was resuspended in 10 mL wash medium and centrifuged as above. The washed cells were then resuspended in RPMI-1640 medium containing 10% FBS to about 6 million cells per mL and 100 mL of the suspension were added into each well of a 96-well microtiter plate and incubated overnight at 37° C. in humidified 5% CO₂. After 24 h the medium was replaced with 200 μL fresh RPMI-1640 medium containing 10% FBS and incubated at 37° C. in humidified 5% CO₂ for 2 days. The peptide aliquot was then added to samples in duplicate at a final concentration of 100 nM and incubated at 37° C. in humidified 5% CO₂ for 4 h. The medium was then removed and stored at −80° C. The samples were analyzed for production of cytokines. One set of control cells was not treated with an experimental agent. A second set of control cells was treated with lipopolysaccharide, an agent commonly used to induce production of a variety of inflammatory cytokines. The positive control for inflammation was essential to ensure that the peptides did not produce an unregulated inflammatory response.

Culture medium was removed for assay of cytokine levels with methods developed by RayBiotech, Inc. (Norcross, Ga.). In this technology, membrane arrays of antibodies against cytokines are incubated with samples of media. After washing, the array was incubated with a cocktail of all antibodies tagged with biotin. The membrane was then washed free of unbound antibodies and incubated with streptavidin, labeled with a fluorescent dye, which binds to biotin. After a final wash, the membrane arrays were read in a fluorescence detector.

The peptides stimulated release of several important cytokines. In particular, IL-21, a cytokine produced by CD4+ T cells that is required for proliferation and differentiation of natural killer cells and CD8+ cytotoxic lymphocytes. Additional cytokines released by the general population of T cells in response to treatment with the peptides of this invention were IFN-γ, IL-4, IL-8, IL-16, IL-17, TNF-β, and RANTES. Of importance, release of the inflammatory cytokines IL-1α, IL-1β, IL-6, IL-10, and TNF-α were not induced. Release of other important cytokines, notably Eotaxin-2, IL-10, IL-13, IL-12p40, and IL-12p70, was reduced (Table 1).

The mixture of cytokines released from PBMCs, in particular T cells, in response to the peptides described herein should provide, either in isolation or in combination with other treatments, an effective modulation of the immune system. Treatment with the peptides of the present invention should induce activation of cells of the immune system in vivo and provide a sustained endogenous elevation of beneficial cytokines, in contrast to the rapid disappearance of these proteins when given exogenously. These cytokine responses are presumably in addition to direct activation of the immune cells engaged in fighting a disease.

TABLE 1
Release of cytokines by PBMC cultures.

| | | |
|---|---|---|
| Cytokine | Source | Activity |

Increased

| | | |
|---|---|---|
| IL-8 | Macrophages | Activation of neutrophils |
| IL-16 | T cells | Lymphocyte chemoattractant |
| IL-17 | T cells | Stimulates secretion of IL-6, IL-8, G-CSF |
| IL-21 | T cells | Mediates innate and adaptive immune |
| | | responses, affects all lymphocytes, |
| | | dendritic cells and monocytes |
| IFN-γ | NK cells | Anti-viral, immunoregulatory, |
| | | anti-tumor properties |
| TNF-β | T cells | Cytolytic or cytostatic for many tumors |
| MIP-1d | T, B, NK cells | Macrophage inflammatory protein, |
| | | activates dendritic cells, granulocytes, |
| | | induces synthesis of pro- |
| | | inflammatory monocytes cytokines |
| RANTES | T cells | Chemotactic for T cells, eosinophils |
| | | and basophils |

Decreased

| | | |
|---|---|---|
| Eotaxin-2 | Dendritic cells, | Chemotaxis of eosinophils, |
| | monocytes | basophils (inflammatory) |
| IL-10 | Monocytes, | Inhibits synthesis of IFN-γ, IL-2 and TNF-β |
| | macrophages | |
| IL-12 | T and B cells, | Activates NK cells, stimulates |
| | macrophages | proliferation of lymphoblasts |
| IL-13 | T cells | Downregulates inflammatory cytokines |

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims. Although the examples herein disclose the therapeutic efficacy of the peptides of the present invention, with respect to neutralizing replication of the HIV virus, for example, the peptides should be useful to treat a wide variety of infections or disorders, including prophylactic treatments for prevention of such maladies, and for enhancing or stabilizing the well-being of healthy subjects.

### Toxicity of Peptides

Human PBMCs were incubated 3 days with peptides and then assay plates were stained with the soluble tetrazolium-based dye MTS to determine cell viability. The mitochondrial enzymes of metabolically active cells metabolize MTS to yield a colored formazan product. After an incubation period of 4 to 6 h at 37° C., the plates were read spectrophotometrically. Cells treated with peptide SVH1C alone or with diluted anti-HIV antiserum were 100±2% viable at peptide concentrations of 1 nM to 1 μM. In another assay to determine cytotoxicity, cells were doubly stained with acridine orange and ethidium bromide. In this assay, viable cells fluoresce green while dead cells fluoresce red. SVH1C did not exhibit cytotoxicity at a concentration of 1 mM, a concentration 10⁶-fold greater than an effective bioactive concentration of 1 nM [38].

Toxicity of the peptide in vivo was tested by injection of a peptide into animals. In preliminary studies on rats, intravenous injections of peptides that provided 1000-fold greater concentrations than an expected therapeutic dose was well tolerated by the animals and no adverse effects of the peptide were been observed. The peptides can be administered in a number of ways, including without limitation by injection (intravenously, subcutaneously, intramuscularly or intraperitoneally, topically (transmucosally, transbuccally, sublingually, or transdermally) and/or orally (liquid, tablet or capsule).

### Evidence for Activation of NK and Cytotoxic T Cells

To explore this phenomenon further, PBMCs were depleted of monocytes by positive selection with magnetic beads to which anti-CD14 antibodies were attached. Depletion of CD14⁺ monocytes was verified by flow cytometry. Cells remaining after the beads were removed were placed in culture and treated as described above. The antibodies were provided as serum from HIV-infected patients by the California Department of Public Health (Richmond, Calif.) and diluted to a concentration which, in the absence of the peptides (data not shown), provided 25% to 40% neutralization. HIV and antisera from HIV-positive individuals were added exactly as described above in the previous section. In an assay in which the PBMCs were depleted of monocytes, inhibition of HIV replication after addition of peptides was 90 to 95%. In this experiment, inhibition without monocytes was considerably greater than before depletion of CD14⁺ cells (about 55%). Thus the dramatic inhibition induced by peptides indicated that an additional cell type other than phagocytes was responsible for the strong inhibition. The additional cell types responsible for the peptide/antibody induced inhibition reside within the lymphocyte population. The most likely cell types are NK cells and cytotoxic T cells, which when activated via receptor NKG2D engage in lysis of infected CD4⁺ cells.

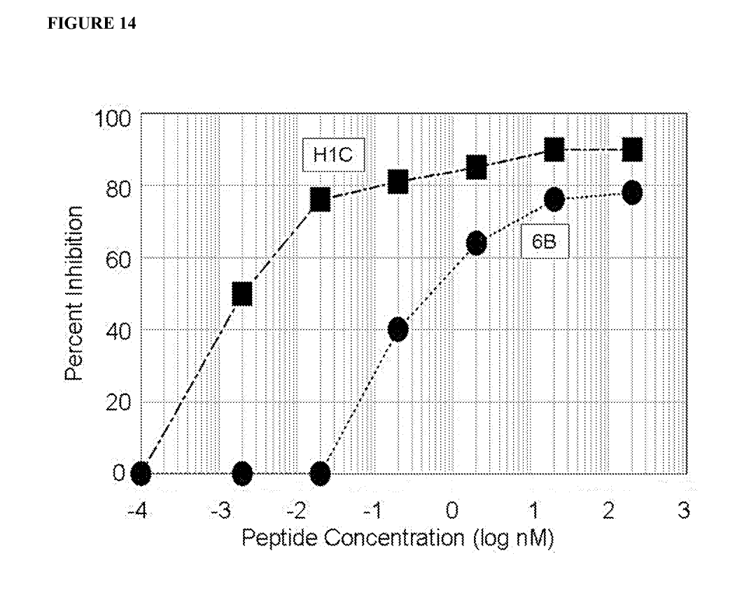

It was found that when the cultures were overwhelmed with a high input of the virus, the percent of neutralization was reduced. Thus, in subsequent experiments the viral input was reduced to about 30 TCID50. An assay was performed in which peptides were added to the culture without antiserum. For the results shown in FIG. 14, the peptides alone inhibited viral replication by 80 to 90%. 1050 values in this experiment were 2 μM for SVH1C and about 300 μM for SV6B. Because antibodies were not present in this experiment, antibody-mediated phagocytosis did not contribute significantly to neutralization. Flow cytometric analysis of the PBMCs indicated a relatively high NK/monocyte ratio.

The data and the observation that addition of monocytes to the assay (other experimental data not shown) did not enhance inhibition by peptides, suggest that non-phagocytic cell types have a prominent role in elimination of HIV-1 from PBMC cultures. The peptides stimulate phagocytic cells [24, 36], and although a clear effect of antiserum is evident, the overall view from the data suggest that NK cells and CD8⁺ cytotoxic T cells are responsible for most of the inhibition of HIV-1 replication, probably by their ability to lyse infected cells. This suggestion is further supported by characteristics of the major activation receptor, NKG2D, on these cells. This protein is a C-type lectin-like receptor that binds Neu5Ac(α2-3)Gal sequences, which are specifically mimicked by SVH1C (FIG. 4). NK cells function as important components of the innate immune system to identify and lyse cells that are stressed by infection or cancer.

Siglecs occur on the surface of most of the cells of the immune system. In general, siglecs show low affinity (a K_{D} of 0.1-3 mM) for the sialic acid Neu5Ac (α2-3) and (α2-6) linkages to galactose [Neu5Acα(2-3)Gal and Neu5Acα(2-6)Gal]. It is assumed that this recognition is important for modulating the functions of siglecs as regulators of adhesion, cell signaling and endocytosis [13, 32].

The fact that the peptides act alone as well as in concert with antibodies against a virus suggest that they may be effective as immune system modulators for use in therapy for a variety of infections against which an individual develops antibodies. The data shown herein demonstrate that the peptides SVH1C (SEQ ID NO:7), SVH1D (SEQ ID NO:8) and SV6B (SEQ ID NO:3), as well as SVC2 (SEQ ID NO:1) which is not shown, functionally mimic glycans with terminal Neu5Ac-Gal sequences. Receptors such as NKG2D and siglecs bind these glycans. Siglec-1 is expressed on monocytes and macrophages and is involved in cellular adhesion but also enhances endocytosis. As such, it enhances infection of these cells by HIV-1 by binding to glycans on the envelop of the virus [33, 34]. Because siglecs are known to bind to the same glycans as the lectins MAA and SNA1 (see FIG. 6), it is expected that peptide SVH1C will also bind to siglecs. Therefore, these peptides should function as modulators of cell activity by serving as a ligand for these receptors. Siglec-1 (aka sialoadhesin) is also highly expressed on inflammatory macrophages from affected tissues in patients with rheumatoid arthritis [38]. The therapeutic peptides and compositions disclosed herein may thus be used to treat or prevent rheumatoid arthritis.

### Synergy Between Antibodies and Peptides

Complete inhibition of HIV-1 replication (neutralization) in PBMCs by several peptide constructs containing four copies of a core sequence linked to a branched central framework structure was demonstrated previously [38]. This peptide construct was assayed for activity with two subtypes of HIV, Glade B (Strain SF162) and Glade C (Strain 97ZA009), both of which were provided by the California Department of Public Health (Richmond, Calif.). HIV-1 Glade B is the major subtype in North America and Europe and HIV-1 Glade C is the major subtype in central and southern Africa, India and China. The peptide was assayed either alone or in combination with serum from HIV-infected individuals. In the absence of peptide, the antibody preparation (serum) provided only 25 to 40% neutralization at the same dilution (data not shown).

It was found that when the cultures were overwhelmed with a high input of the virus, the percent of neutralization was reduced. Thus, in subsequent experiments the viral input was reduced to about 30 TCID50. An assay was performed in which peptides were added to the culture without antiserum. For the results shown in FIG. 14, the peptides alone inhibited viral replication by 80 to 90%. 1050 values in this experiment were 2 μM for SVH1C and about 300 μM for SV6B. Because antibodies were not present in this experiment, antibody-mediated phagocytosis did not contribute significantly to neutralization. Flow cytometric analysis of the PBMCs indicated a relatively high NK/monocyte ratio. The peptides stimulated phagocytic cells [16, 38], and although a clear effect of antiserum is evident, the overall view from the data suggest that NK cells and CD8⁺ cytotoxic T cells are responsible for most of the inhibition of HIV-1 replication, probably by their ability to lyse infected cells. This suggestion is further supported by characteristics of the major activation receptor, NKG2D, on these cells. This protein is a C-type lectin-like receptor that binds Neu5Ac(α2-3)Gal sequences, which are specifically mimicked by NPSHPLSG (SEQ ID NO:7; FIG. 4). NK cells function as important components of the innate immune system to identify and lyse cells that are stressed by infection or cancer.

### SUMMARY

The fact that the peptides act alone as well as in concert with antibodies against a virus suggest that they may be effective as immune system modulators for use in therapy for a variety of infections against which an individual develops antibodies. The data shown herein demonstrate that the peptides SVH1C (NPSHPLSG, SEQ ID NO:7), SVH1D (NPSHPSLG, SEQ ID NO:8) and SV6B (HPSLK, SEQ ID NO:3) functionally mimic glycans with terminal Neu5Ac-Gal sequences. Receptors such as NKG2D and siglecs bind these glycans. Siglec-1 is expressed on monocytes and macrophages and is involved in cellular adhesion but also enhances endocytosis of viruses [35, 36]. Therefore, these peptides should function as modulators of cell activity by serving as a ligand for these receptors.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

### REFERENCES

1. Miyauchi K, Kim Y, Latinovic 0, Morozov V, Melikyan G B. 2009. HIV enters cells via endocytosis and dynamin-dependent fusion with endosomes. Cell 137:433-444.
2. Choudhary S K, Margolis D M. 2011. Curing HIV: Pharmacologic approaches to target HIV-1 latency. Annu Rev Pharmacol Toxicol 51:397-418.
3. Welch B D, Francis J N, Redman J S, Paul S, Weinstock M T, Reeves J D, Lie Y S, Whitby F G, Eckert D M, Hill C P, Root M J, Kay M S. 2010. Design of a potent D-peptide HIV-1 entry inhibitor with a strong barrier to resistance. J Virol 84:11235-11244.
4. Korber B, Gnanakaran S. 2011. Converging on an HIV vaccine. Science 333:1589-1590.
5. Davey R T, Bhat N, Yoder C, and 17 co-authors. 1999. HIV-1 and T cell dynamics after interruption of highly active antiretroviral therapy (HAART) in patients with a history of sustained viral suppression. Proc Natl Acad Sci USA 96:15109-15114.
6. US Food and Drug Administration. May 23, 2011. Approval of Victrelis (boceprevir), a direct acting antiviral drug (DAA) to treat hepatitis C virus (HCV); Approval of Incivek (telaprevir), a direct acting antiviral drug (DAA) to treat hepatitis C virus (HCV).
7. Eggink L L, Hoober J K. 2009. A biologically active peptide mimetic of N-acetylgalactosamine/galactose. BMC Res Notes 2:23.
8. Yi J S, Du M, Zajac A J. 2009. A vital role for interleukin-21 in the control of chronic viral infection. Science 324:1572-1576.
9. Elsaesser H, Sauer K, Brooks D G. 2009. IL-21 is required to control chronic viral infections. Science 324:1569-1572.
10. Raulet D H. 2003. Roles of the NKG2D immunoreceptor and its ligands. Nature Rev Immunol 3:781-790.
11. Iannello A, Debbeche O, Samarani S, Ahmad A. 2008. Antiviral NK cell responses in HIV infection: 1. NK cell receptor genes as determinants of HIV resistance and progression to AIDS. J Leukoc Biol 84:1-26.
12. Macauley M S, Crocker P R, Paulson J C. 2014. Siglec-mediated regulated of immune cell function in disease. Nature Rev Immunol 14: 653-666.
13. Jellusova J, Nitschke L. 2012. Regulation of B cell functions by the sialic acid-binding receptors Siglec-G and CD22. Front Immunol 2: Article 96.
14. Prescher H, Schweizer A, Kuhfeldt E, Nitschke L, Brossmer R. 2014. Discovery of multifold modified sialosides as human CD22/Siglec-2 ligands with nanomolar activity on B-cells. ACS Chem Biol 9: 1444-1450.
15. Imaizumi Y, Higai K, Suzuki C, Azuma Y, Matsumoto K. 2009. NKG2D and CD94 bind to multimeric α2,3-linked N-acetylneuraminic acid. Biochem Biophys Res Commun 382:604-608.
16. Eggink L L, Hoober J K. 2010. Peptide mimetics of terminal sugars of complex glycans. Glycobiol Insights 2:1-12.
17. Champsaur M, Lanier L L. 2010. Effect of NKG2D ligand expression on host immune responses. Immunol Rev 235:267-285.
18. Geijtenbeek T B H, Gringhuis S I. 2009. Signalling through C-type lectin receptors: shaping immune responses. Nature Rev Immunol 9:465-479.
19. Garcia-Vallejo J J, van Kooyk Y. 2009. Endogenous ligands for C-type lectin receptors: the true regulators of immune homeostasis. Immunol Rev 230:22-37.
20. Bone H, Williams N A. 2001. Antigen-receptor cross-linking and lipopolysaccharide trigger distinct phosphoinositide 3-kinase-dependent pathways to NK-κB activation in primary B cells. Int Immunol 13:807-816.
21. Marsh C B, Lowe M P, Rovin B H, Parker J M, Liao Z, Knoell D L, Wewers M D. 1998.
Lymphocytes produce IL-1 beta in response to Fcgamma receptor cross-linking: effects on parenchymal cell IL-8 release. J Immunol 160:3942-3948.
22. Li P, Morris D L, Willcox B E, Steinle A, Spies T, Strong R K. 2001. Complex structure of the activating immunoreceptor NKG2D and its MHC class 1-like ligand MICA. Nature Immunol 2:443-451.
23. McFarland B J, Kortemme T, Yu S F, Baker D, Strong R K. 2003. Symmetry recognizing asymmetry: analysis of the interactions between the C-type lectin-like immunoreceptor NKG2D and MHC class 1-like ligands. Structure 11:411-422.
24. Mammen M, Choi S-K, Whitesides G M. 1998. Polyvalent interactions in biological systems: implications for design and use of multivalent ligands and inhibitors. Angew Chem Int Ed 37:2754-2794.
25. Dimick S M, Powell S C, McMahon S A, Moothoo D N, Naismith J H, Toone E J. 1999. On the meaning of affinity: cluster glycoside effects and Concanavalin A. J Am Chem Soc 121:10286-10296.
26. Cairo C W, Gestwicki J E, Kanai M, Kiessling L L. 2002. Control of multivalent interactions by binding epitope density. J Am Chem Soc 124:1615-1619.
27. Knibbs R N, Goldstein I J, Ratcliffe R M, Shibuya N. 1991. Characterization of the carbohydrate binding specificity of the leukoagglutinating lectin from Maachia amurensis. J Biol Chem 266:83-88.
28. Shibuya N, Goldstein I J, Broekaert W F, Nsimba-Lubaki M, Peeters B, Peumans W J. 1987. The elderberry (Sambucus nigra L.) bark lectin recognizes the Neu5Ac(α2-6)Gal/GalNAc sequence. J Biol Chem 262:1596-1601.
29. Spiro R G, Bhoyroo V D. 1974. Structure of the O-glycosidically linked carbohydrate units of fetuin. J Biol Chem 249:5704-5717.
30. Baenziger J U, Fiete D. 1979. Structure of the complex oligosaccharides of fetuin. J Biol Chem 254:789-795.
31. Blixt O, Han S, Liao L et al. 2008. Sialoside analogue arrays for rapid identification of high affinity siglec ligands. J Am Chem Soc 130:6680-6681.
32. Lakey J H, Raggett E M. 1998. Measuring protein-protein interactions. Curr Opin Struct Biol 8:119-123.
33. Bülow A, Plesner I W, Bols M. 2000. A large difference in the thermodynamics of binding of isofagomine and 1-deoxynojirimycin to β-glucosidase. J Am Chem Soc 122:8567-8568.
34. O'Reilly M K, Paulson J C. 2009. Siglecs as targets for therapy in immune-cell-mediated disease. Trends Pharmacol Sci 30:240-248.
35. Rempel H, Calosing C, Sun B, Pulliam L. 2008. Sialoadhesin expressed on IFN-induced monoctes binds HIV-1 and enhances infectivity. PLoS ONE 3(4):e1967.
36. Zou Z, Chastain A, Moir S and 7 co-authors. 2011. Siglecs facilitate HIV-1 infection of macrophages through adhesion with viral sialic acids. PLoS ONE 6(9):e24559.
37. Angata T, Tabuchi Y, Nakamura K, Nakamuras M. 2007. Siglec-15: an immune system Siglec conserved throughout vertebrate evolution. Glycobiology 17:838-846.
38. Eggink L L, Salas M, Hanson C V, Hoober J K. 2010. Peptide sugar mimetics prevent HIV-1 replication in peripheral blood mononuclear cells in the presence of HIV-positive antiserum. AIDS Res Human Retrovir 26:149-160.

What is claimed is:

1. A method of stimulating the immune system of an immunocompromised subject, the method comprising: administering to the immunocompromised subject a composition comprising a therapeutic peptide or a multivalent structured polypeptide comprising multiple copies of the therapeutic peptide, the therapeutic peptide consisting of 5 to 8 amino acids and selected from the group consisting of:
(SEQ ID NO: 1)
VGGGS
and
X1-P-S-X2-X3-X4-X5-X6,
wherein
X1 is selected from the group consisting of H and N, or is absent;
X2 is selected from the group consisting of L, S, N, and H;
X3 is selected from the group consisting of N, K, G, L, P, and A;
X4 is selected from the group consisting of A, S, and L, or is absent;
X5 is selected from the group consisting of S and L, or is absent; and
X6 is G, or is absent;
wherein the therapeutic peptide or multivalent structured polypeptide is in an amount sufficient to activate and/or stimulate proliferation of immune cells in the subject.

2. The method of claim 1, wherein the therapeutic peptide or multivalent structured polypeptide stimulates proliferation of immune cells selected from the group consisting of macrophages; dendritic cells; natural killer cells; natural killer T cells; CD3⁺, CD4⁺ and CD8⁺ T cells; B cells; and combinations thereof.

3. The method of claim 2, wherein the immune cells are memory B cells.

4. The method of claim 1, further comprising determining (a) the level of immune cells in the immunocompromised subject before administering the composition; and (b) the level of immune cells in the immunocompromised subject after administering the composition.

5. The method of claim 4, wherein (a) and (b) are determined with flow cytometry.

6. The method of claim 4, wherein the ratio of (b) to (a) is at least 2, at least 3, at least 4, or at least 5.

7. The method of claim 1, wherein the therapeutic peptide or multivalent structured polypeptide activates the immune cells by binding to:
i) an activating receptor NKG2D; and/or
ii) an inhibitory receptor sialic acid-binding immunoglobulin-like lectin (siglec).

8. The method of claim 1, wherein the therapeutic peptide is selected from the group consisting of: VGGGS (SEQ ID NO:1), PSSNA (SEQ ID NO:2), HPSLK (SEQ ID NO:3), NPSHPLSG (SEQ ID NO:7), and NPSHPSLG (SEQ ID NO:8).

9. The method of claim 1, wherein the multivalent structured polypeptide is branched.

10. The method of claim 1, wherein the therapeutic peptide functionally mimics a terminal sequence 5-acetylneuraminic acid-galactose or N-acetylgalactosamine on complex glycans, the terminal sequence being linked α(2-3) or α(2-6).

11. A method of immunotherapy comprising: administering to a subject with cancer and/or a viral infection a composition comprising a therapeutic peptide or a multivalent structured polypeptide comprising multiple copies of the therapeutic peptide, the therapeutic peptide consisting of 5 to 8 amino acids and selected from the group consisting of:
(SEQ ID NO: 1)
VGGGS
and
X1-P-S-X2-X3-X4-X5-X6,
wherein
X1 is selected from the group consisting of H and N, or is absent;
X2 is selected from the group consisting of L, S, N, and H;
X3 is selected from the group consisting of N, K, G, L, P, and A;
X4 is selected from the group consisting of A, S, and L, or is absent;
X5 is selected from the group consisting of S and L, or is absent; and
X6 is G, or is absent;
wherein the therapeutic peptide or multivalent structured polypeptide is in an amount sufficient to activate and/or stimulate proliferation of immune cells in the subject.

12. The method of claim 11, wherein the therapeutic peptide or multivalent structured polypeptide stimulates proliferation of immune cells selected from the group consisting of macrophages; dendritic cells; natural killer cells; natural killer T cells; CD3⁺, CD4⁺ and CD8⁺ T cells; B cells; and combinations thereof.

13. The method of claim 11, wherein the therapeutic peptide or multivalent structured polypeptide activates the immune cells by binding to:
i) an activating receptor NKG2D; and/or
ii) an inhibitory receptor sialic acid-binding immunoglobulin-like lectin (siglec).

14. The method of claim 11, further comprising determining (a) the level of immune cells in the immunocompromised subject before administering the composition; and (b) the level of immune cells in the immunocompromised subject after administering the composition.

15. The method of claim 14, wherein (a) and (b) are determined with flow cytometry.

16. The method of claim 14, wherein the ratio of (b) to (a) is at least 2, at least 3, at least 4, or at least 5.

17. The method of claim 11, wherein the therapeutic peptide is selected from the group consisting of: VGGGS (SEQ ID NO:1), PSSNA (SEQ ID NO:2), HPSLK (SEQ ID NO:3), NPSHPLSG (SEQ ID NO:7), and NPSHPSLG (SEQ ID NO:8).

18. The method of claim 11, wherein the subject suffers from an HIV infection.

19. The method of claim 11, wherein the subject suffers from cancer.
